(19) United States
(12) Patent Application Publication
(10) Pub. No.: US 2024/0138865 A1
(43) Pub. Date: May 2, 2024

(54) METHOD AND APPARATUS FOR ENDOSCOPIC STONE RETRIEVAL

(71) Applicant: EndoTheia, Inc., Nashville, TN (US)

(72) Inventors: Joshua Gafford, Nashville, TN (US); Scott Webster, Nashville, TN (US); Patrick Anderson, Nashville, TN (US); Caleb Rucker, Knoxville, TN (US); Robert Webster, Nashville, TN (US)

(73) Assignee: EndoTheia, Inc., Nashville, TN (US)

(21) Appl. No.: 18/238,258

(22) Filed: Aug. 25, 2023

Related U.S. Application Data
(60) Provisional application No. 63/419,842, filed on Oct. 27, 2022.

(51) Int. Cl. A61B 17/221

(52) U.S. Cl. CPC A61B 17/221; A61B 2017/00367

(57) ABSTRACT

The present disclosure provides for an endoscopic apparatus and methods. The apparatus may include a steerable sheath, a control wire disposed within the steerable sheath, and a basket disposed on the control wire. The steerable sheath may be actuated to form a bend, advancing movements of the control wire may cause the basket to project out of the steerable sheath and expand, and retreating movements of the control wire may cause the basket to retract into the steerable sheath and contract.

### CROSS-REFERENCE TO RELATED APPLICATIONS

This application is a non-provisional application and claims priority to U.S. Provisional Patent Application No. 63/419,842, filed Oct. 27, 2022, entitled METHOD AND APPARATUS FOR ENDOSCOPIC STONE RETRIEVAL, which is hereby incorporated by reference in its entirety.

### FEDERALLY SPONSORED RESEARCH OR DEVELOPMENT

This invention was made with government support under grant number 1R44EB031741-01 awarded by the National Institutes of Health (NIH). The government has certain rights in the invention.

### BACKGROUND

The present disclosure relates generally to devices and methods for endoscopic surgery, and more particularly to providing endoscopic devices for performing minimally invasive ureteroscopic kidney stone removal within a patient.

Each year three-and-a-half million people suffer from kidney stones, with one out of every five (approximately seven-hundred thousand) requiring an intervention. Of these seven-hundred thousand patients, sixty-three percent have small stones and are well-served by the present standard of care, which often includes flexible ureteroscopy. The typical ureteroscopy procedure includes an endoscope being passed through the urethra, bladder, and then directly into the upper urinary tract in order to remove a kidney stone. Depending on the size of the stone to be removed, ureteroscopy procedures may require a laser lithotripsy procedure (e.g., using a laser to break kidney stones into tiny pieces) via the endoscope before deploying a flexible ureteroscopic kidney stone basket from the endoscope to capture and remove the pieces using the endoscope. As the size of the kidney stones get larger, however, surgeons face a troubling dilemma in how to treat the remaining thirty-seven percent of patients (approximately two-hundred and sixty-thousand per year in the U.S.A. alone) who have larger stones (e.g., eleven millimeters or larger in diameter) that make typical procedures difficult and inefficient. For example, dexterity limitations in aiming the laser for performing laser lithotripsy and capturing the pieces of the stone using the basket make procedures involving larger stone relatively long in duration (e.g., exceeding two hours) and highly variable (e.g., risking complications involved in the extended presence of the endoscope within the patient). This is particularly true in lower-pole cases (e.g., cases involving stones located in the lower calyx of the kidney), which may be the most common renal stone that patients incur. In such cases, due to the anatomical location of the kidney stone, the aforementioned lack of dexterity makes it extremely challenging to capture all stones with the basket effectively.

Many such baskets have been developed to enable the capture of renal calculi, including standard “tipped” baskets, “tip-less” designs, and “end-engaging” designs. All of these baskets operate through the standard principle of positioning a basket that is in a “released” configuration about a stone (e.g., such that the stone is within the bounds of a number of pre-curved wires making up the basket), transitioning the basket into “collapsed” configuration by retracting pre-curved wires into an outer tube or collection of tubes to entrap the stone, and withdrawing the basket from the patient. A drawback of the prior art is the fact that the basket can only be inserted into and retracted from the surgical field in a linear fashion, which lends to the issues surrounding dexterity mentioned above. This is problematic especially in the lower calyces where stones may be located around a corner in a calyx that cannot be accessed directly by the ureteroscope. In this situation, the physician must try to passively deflect the basket into the calyx by “bouncing” the basket cage off of the tissue to try and re-direct the basket into the calyx. This can be an extremely cumbersome and frustrating process that adds substantially to procedure time and can lead to patient trauma due to the repeated interaction between the basket and patient tissue.

What is needed, then, are improvements in devices and methods for ureteroscopic kidney stone removal within a patient.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is an apparatus for removing an anatomical object (such as a kidney stone) from within a patient by deploying a basket from a steerable sheath that may be inserted into the patient. For example, the apparatuses and methods described herein may provide a method of removing kidney stones, and other anatomical objects that may need to be removed from the patient, with improved dexterity afforded by the steerable sheath. The apparatuses described herein may thus facilitate kidney stone removal with an improved procedure time and less complications.

The apparatus includes a steerable sheath (e.g., a tube, arm, tool, member, etc.) through which a basket, and a control wire coupled thereto, may extend. The steerable sheath may be actuated to form a bend in order to steer a distal end of the steerable sheath, thus maneuvering a basket located thereon once deployed through the distal tip into an expanded configuration, which may be used to capture the object before being retracted through the inner lumen of the steerable sheath into a collapsed state that retains the object. The steerable sheath, then, may be retracted from the patient, thus removing the object.

In some embodiments, the steerable sheath has a concentric tube structure including nested concentric tubes, such that the steerable sheath is actuable to form a bend through applying an axial force to the concentric tubes. The control wire may be disposed within the steerable sheath and be movable therein along a longitudinal axis of the steerable sheath. The basket, which may be disposed on the control wire, includes a number of wires. Advancing movements of the control wire thus cause the basket to project out of an opening formed by the distal end of the steerable sheath and expand. Retreating movements of the control wire, on the other hand, cause the basket to retract into the opening and contract.

Another embodiment of the present disclosure is a method of retrieving an anatomical object from within a patient. A steerable sheath is provided with a basket coupled to a control wire that is movable along a longitudinal axis of the steerable sheath. By actuating the steerable sheath to form a bend and expanding and/or contracting the basket via advancements and/or retractions of the control wire (respectively), the anatomical object, which may be a kidney stone is retained by the basket.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a perspective view of a tube assembly for an apparatus for endoscopic stone removal, according to some embodiments of the present disclosure.

FIG. 2 is a perspective view a method of assembling the tube assembly of FIG. 1, according to some embodiments of the present disclosure.

FIG. 3A is a side view of embodiments of a first flexible tube and a second flexible tube that are assembled to construct a steerable sheath for endoscopic stone retrieval, according to some embodiments of the present disclosure.

FIG. 3B is a side view of the first flexible tube and the second flexible tube of FIG. 3A assembled to form the steerable sheath, according to some embodiments.

FIG. 3C is a side view of the assembled steerable sheath of FIG. 3B being actuated to form a bend in a first direction, according to some embodiments of the present disclosure.

FIG. 3D is a side view of the assembled steerable sheath of FIG. 3B being actuated for form a bend in a second direction, according to some embodiments of the present disclosure.

FIG. 3E is a perspective view of a tube assembly for an apparatus for endoscopic stone removal, where a steerable sheath of the apparatus is being actuated to form a bend in a first direction, according to some embodiments of the present disclosure.

FIG. 3F is a perspective view of a tube assembly for an apparatus for endoscopic stone removal, where a steerable sheath of the apparatus is being actuated to form a bend in a second direction, according to some embodiments of the present disclosure.

FIG. 4A is a side cross-sectional view of a tube assembly for an apparatus for endoscopic stone removal, where a steerable sheath of the apparatus is being actuated to form a bend in a first direction, according to some embodiments of the present disclosure.

FIG. 4B is a side cross-sectional view of a tube assembly for an apparatus for endoscopic stone removal, where a steerable sheath of the apparatus is being actuated to form a bend in a second direction, according to some embodiments of the present disclosure.

FIG. 4C is an axial cross-sectional view of a tube assembly for an apparatus for endoscopic stone removal, according to some embodiments of the present disclosure.

FIG. 5A is a side view of a first flexible tube and second flexible tube for a steerable sheath, according to some embodiments of the present disclosure.

FIG. 5B is a side view of a first flexible tube and second flexible tube for a steerable sheath, according to some embodiments of the present disclosure.

FIG. 5C is a side view of a first flexible tube and second flexible tube for a steerable sheath, according to some embodiments of the present disclosure.

FIG. 5D is a side view of a tube assembly for an apparatus for endoscopic stone removal, where a steerable sheath of the apparatus is being actuated to form a bend in a first direction, according to some embodiments of the present disclosure.

FIG. 5E is a side view of a tube assembly for an apparatus for endoscopic stone removal, where a steerable sheath of the apparatus is being actuated to form a bend in a second direction, according to some embodiments of the present disclosure.

FIG. 5F is a side view of a tube assembly for an apparatus for endoscopic stone removal, where a steerable sheath of the apparatus is being actuated to form a bend in a second direction, according to some embodiments of the present disclosure.

FIG. 6A is a detailed perspective view of a tube assembly for endoscopic stone removal, according to some embodiments of the present disclosure

FIG. 6B is a perspective view of the tube assembly of FIG. 6A, shown capturing a kidney stone using a basket of the tube assembly, according to some embodiments of the present disclosure.

FIG. 7A is a perspective view of a basket for endoscopic stone removal in a collapsed state, according to some embodiments of the present disclosure.

FIG. 7B is a perspective view of the basket of FIG. 7A in a released state, according to some embodiments of the present disclosure.

FIG. 7C is a perspective view of a tube assembly for endoscopic stone removal advancing a basket into a released state, according to some embodiments of the present disclosure.

FIG. 7D is a perspective view of a tube assembly for endoscopic stone removal retreating a basket into a collapsed state, according to some embodiments of the present disclosure.

FIG. 7E is a perspective view of a tube assembly for endoscopic stone removal advancing a basket into a released state, according to some embodiments of the present disclosure.

FIG. 7F is a perspective view of a tube assembly for endoscopic stone removal retreating a basket into a collapsed state, according to some embodiments of the present disclosure.

FIG. 8A is a schematic view of the apparatus for endoscopic stone removal, shown retrieving a kidney stone from within the anatomy of a patient, according to some embodiments.

FIG. 8B is a detailed perspective view of a basket of the apparatus of FIG. 8A, according to some embodiments of the present disclosure.

FIG. 9A is a perspective view of an apparatus for endoscopic stone removal, shown with a user interface coupled to a tube assembly, according to some embodiments of the present disclosure.

FIG. 9B is a side cross-sectional view of the apparatus of FIG. 9A, according to some embodiments of the present disclosure.

FIG. 9C is a side cross-sectional view of the apparatus of FIG. 9A, according to some embodiments of the present disclosure.

FIG. 10 is a perspective view of an apparatus for endoscopic stone removal, shown with a user interface coupled to a tube assembly and a ureteroscope, according to some embodiments of the present disclosure.

FIG. 11A is a perspective view of the apparatus of FIG. 9A, according to some embodiments of the present disclosure.

FIG. 11B is a perspective view of the apparatus of FIG. 9A being actuated to advance a basket, according to some embodiments of the present disclosure.

FIG. 11C is a perspective view of the apparatus of FIG. 9A where a steerable sheath of the apparatus is being actuated to form a bend in a first direction, according to some embodiments of the present disclosure.

FIG. 11D is a perspective view of the apparatus of FIG. 9A where a steerable sheath of the apparatus is being actuated to form a bend in a first direction, according to some embodiments of the present disclosure.

FIG. 11E is a perspective view of the apparatus of FIG. 10 being actuated to advance a basket, according to some embodiments of the present disclosure.

FIG. 11F is a perspective view of the apparatus of FIG. 10 where a steerable sheath of the apparatus is being actuated to form a bend in a first direction, according to some embodiments of the present disclosure.

FIG. 11G is a perspective view of the apparatus of FIG. 10 where a steerable sheath of the apparatus is being actuated to form a bend in a first direction, according to some embodiments of the present disclosure.

FIG. 11H is a perspective view of the apparatus of FIG. 10 where the user interface is being rotated to rotate a basket, according to some embodiments of the present disclosure.

FIG. 12A is a side cross-sectional view of a user interface for endoscopic stone removal, according to some embodiments of the present disclosure.

FIG. 12B is a side view of the user interface of FIG. 12A being engaged with a ureteroscope, according to some embodiments of the present disclosure.

FIG. 12C is a side view of the user interface of FIG. 12A being advanced to engage with a ureteroscope, according to some embodiments of the present disclosure.

FIG. 12D is a side view of the user interface of FIG. 12A being engaged with a ureteroscope, according to some embodiments of the present disclosure.

FIG. 12E is a side view of the user interface of FIG. 12A being engaged with a ureteroscope, according to some embodiments of the present disclosure.

FIG. 12F is a perspective view of the apparatus of FIG. 10 where the user interface is being pressed towards the ureteroscope to project a steerable sheath from a ureteroscope tube, according to some embodiments of the present disclosure.

FIG. 13A is a side cross-sectional view of an alternative user interface for an apparatus for endoscopic stone removal, where a lever is being actuated to cause a steerable sheath of the apparatus to form a bend in a first direction, according to some embodiments of the present disclosure.

FIG. 13B is a detailed perspective view of the steerable sheath of FIG. 13A, according to some embodiments of the present disclosure.

FIG. 13C is a side cross-sectional view of an alternative user interface for an apparatus for endoscopic stone removal, where a lever is being actuated to cause a steerable sheath of the apparatus to form a bend in a second direction, according to some embodiments of the present disclosure.

FIG. 13D is a detailed perspective view of the steerable sheath of FIG. 13C, according to some embodiments of the present disclosure.

FIG. 13E is a side cross-sectional view of an alternative user interface for an apparatus for endoscopic stone removal, where a trigger is being actuated to advance a basket, according to some embodiments of the present disclosure.

FIG. 13F is a detailed perspective view of the steerable sheath of FIG. 13E, according to some embodiments of the present disclosure.

FIG. 14A is a perspective view of a rigid apparatus for endoscopic stone removal, according to some embodiments of the present disclosure.

FIG. 14B is a detailed perspective view of a steerable sheath of FIG. 14A, according to some embodiments of the present disclosure.

FIG. 15 is a perspective view of a robotic actuator for an apparatus for endoscopic stone removal, according to some embodiments of the present disclosure.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

Referring generally to the FIGURES, the present disclosure provides for an apparatus for retrieving a stone (e.g., calculi, kidney stone, gallbladder stone, etc.) from within a patient (apparatus) 100. In various embodiments, such apparatuses include a tube assembly 10, along with a steerable sheath 12 incorporated therein. As described herein, the steerable sheath 12 may be configured as an agonist-antagonist concentric tube manipulator, and thus be actuable (e.g., controllable displaceable, translatable, rotatable, configured to be actuated, able to be actuated, etc.) to form a bend (e.g., a curve, deflection, etc.) such that a distal end 22 of the steerable sheath 12 is steered (e.g., deflected) toward an anatomical region (e.g., a surgical site) in order to perform stone retrieval as provided for herein. For example, the steerable sheath 12 may include a nested concentric tube structure including a first flexible tube 28 concentrically nested within (e.g., concentrically disposed within) a second flexible tube 26. Each of the first and second flexible tubes 28, 26 may include a rigid portion along its longitudinal length and a flexible portion along its longitudinal length. In further embodiments, the entire length of each of the first and second flexible tubes 28, 26 is rigid exception for a flexible portion at or near the distal end 22 of the steerable sheath 12. As such, each of the first and second flexible tubes 28, 26 may be flexible in that they each include a flexible portion. As described in greater detail below, each of the first and second flexible tubes 28, 26 may be configured to feature asymmetric flexibility in order in order to employ a concentric agonist-antagonist actuation scheme.

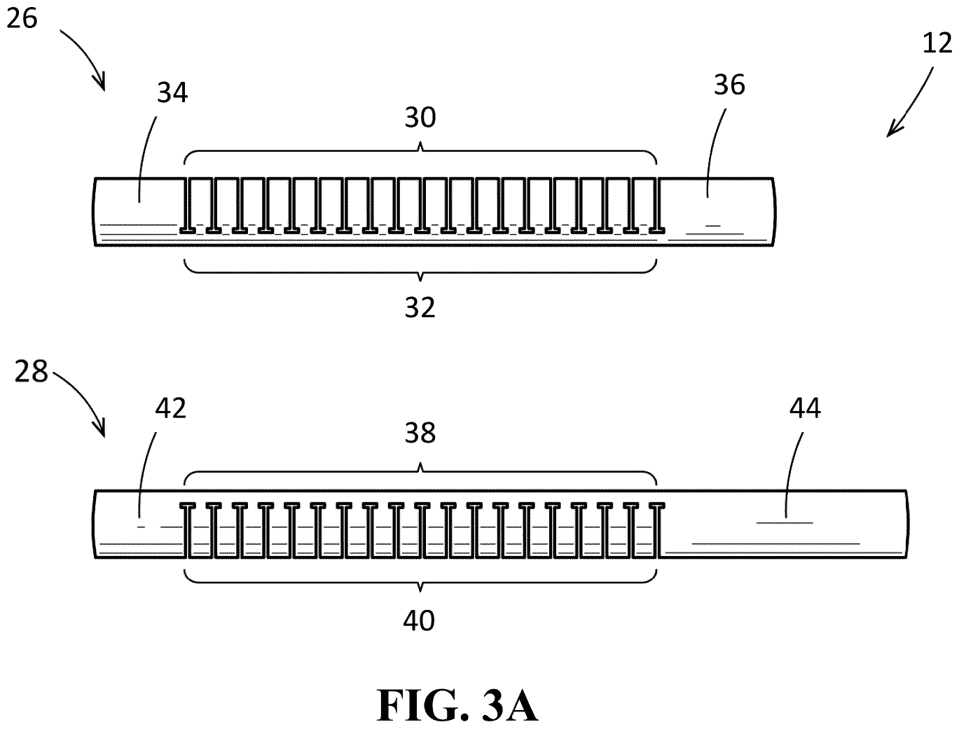

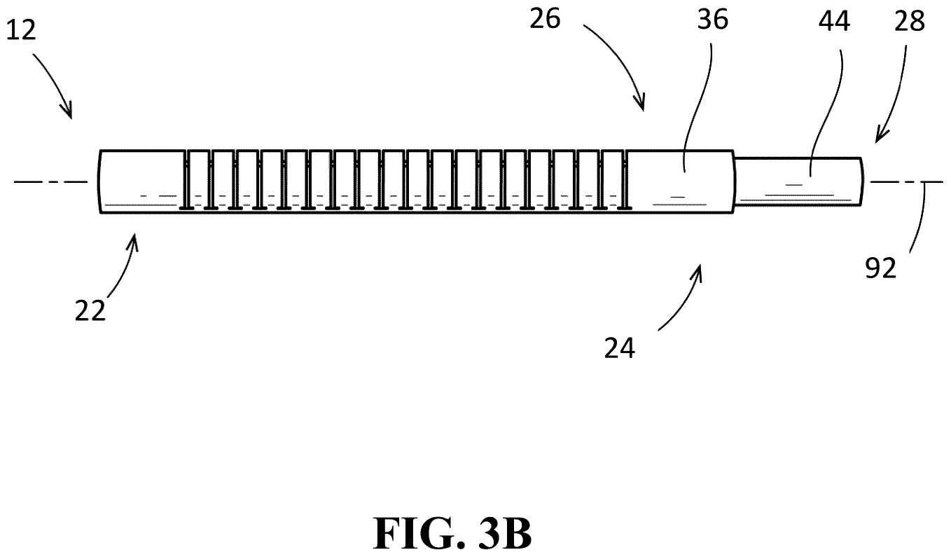

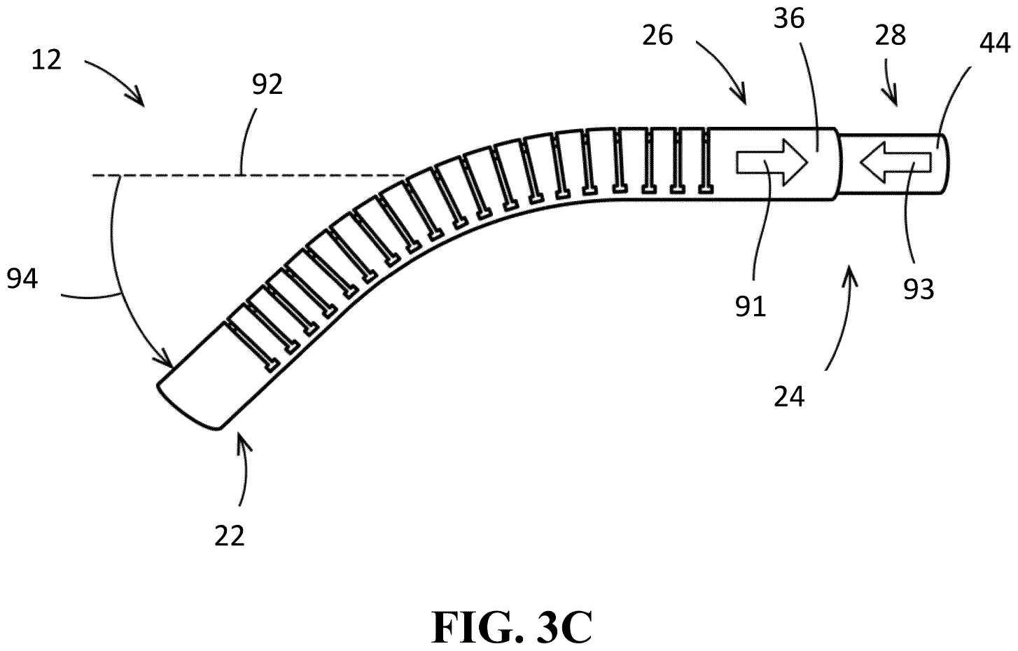

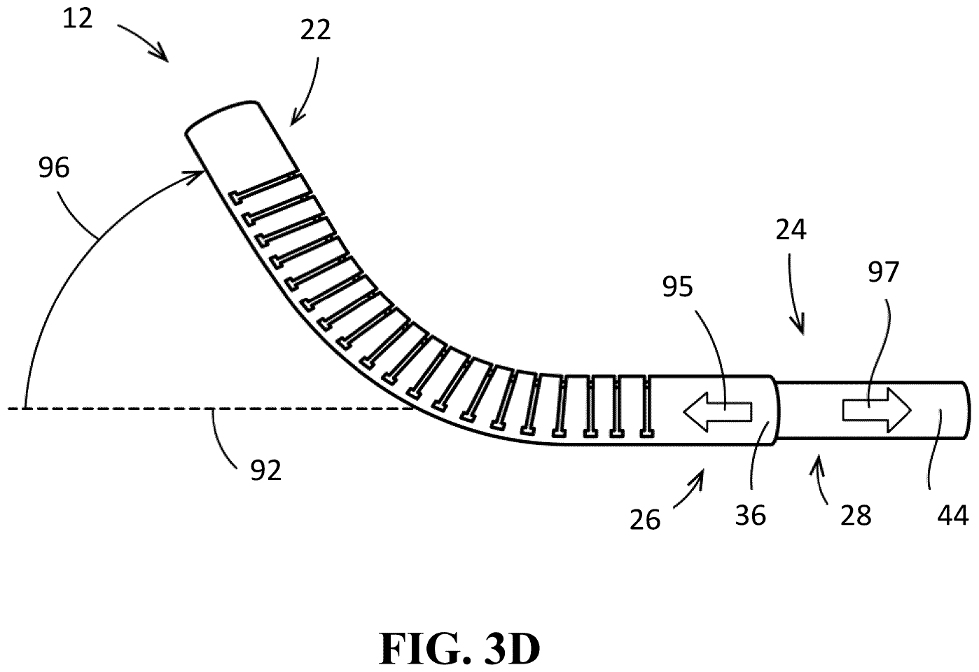

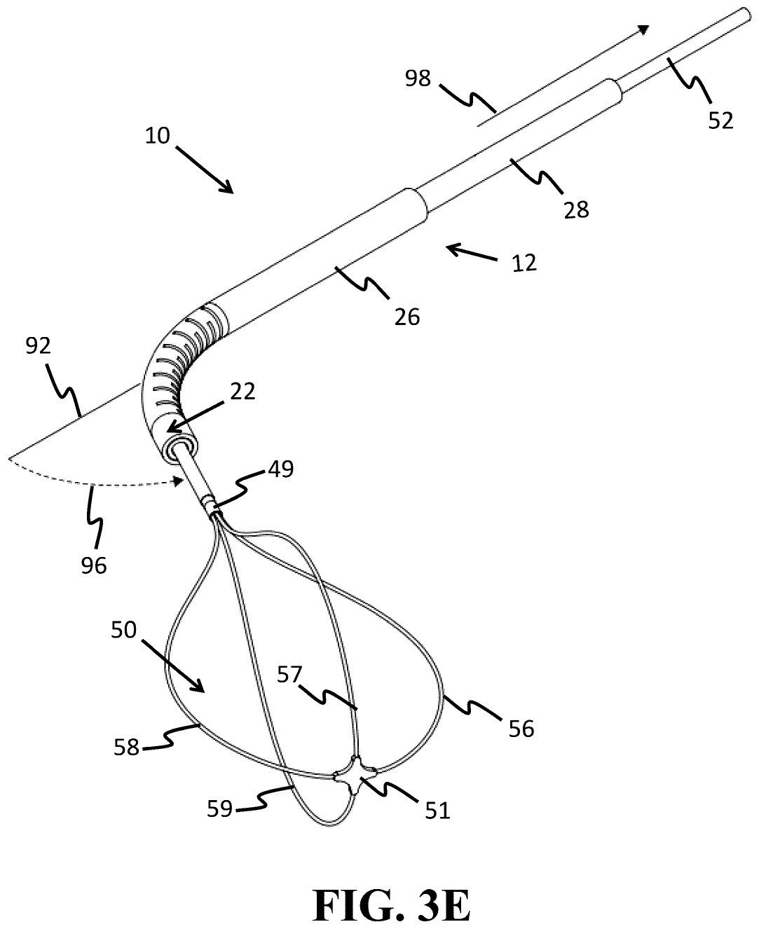

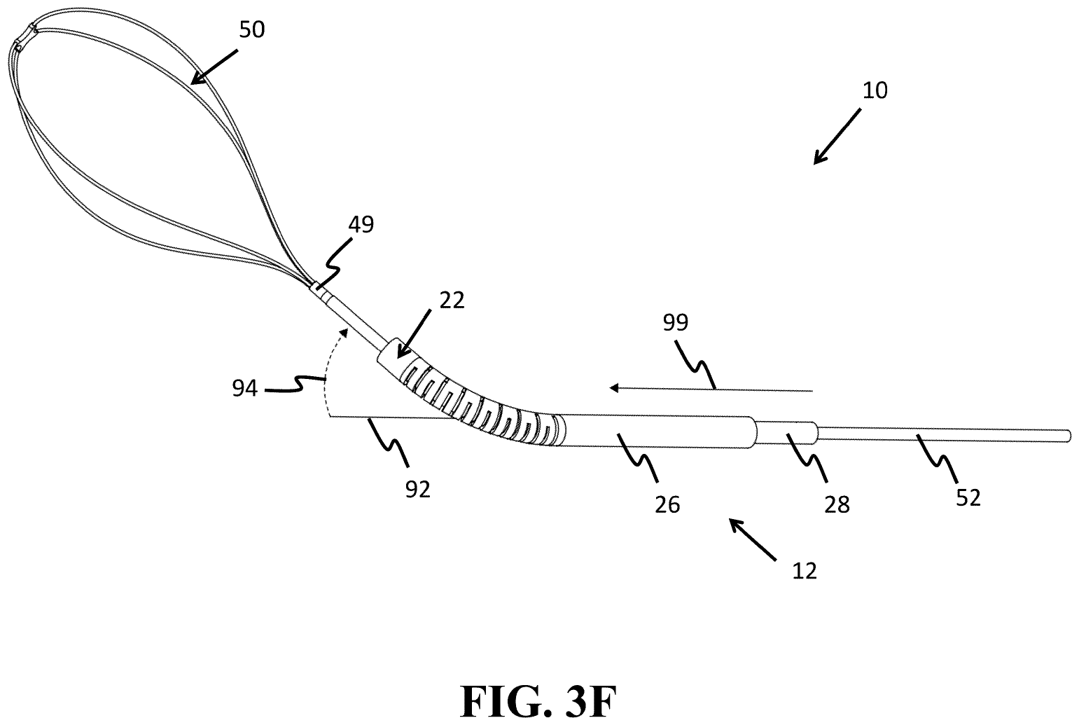

In some embodiments, the tube assembly 10 further includes a basket 50 coupled to a control wire 52, which may be operated to retrieve a stone. The control wire 52 may be disposed within the steerable sheath 12 and be movable therein along a longitudinal axis 92 of the steerable sheath 12 (shown with reference to FIGS. 3C and 3D). For instance, at least a portion of the control wire 52 may be disposed within an inner lumen the steerable sheath 12. The steerable sheath 12 may thus be utilized to control the position of the basket 50 for retrieving a stone. For instance, the basket 50 may be disposed on the control wire 54 and be positioned at least partially within the steerable sheath 12. Accordingly, the apparatus 100 may be utilized as a minimally invasive medical device for removing stones from the renal, ureteral, or biliary systems of a patient. While generally described in the context of removing stones from within a kidney (and the calyces located therein) of a patient, it should be appreciated that the apparatuses and methods described herein may be applied to the retrieval of a wide variety of anatomical objects within various anatomical settings, structures, and situations.

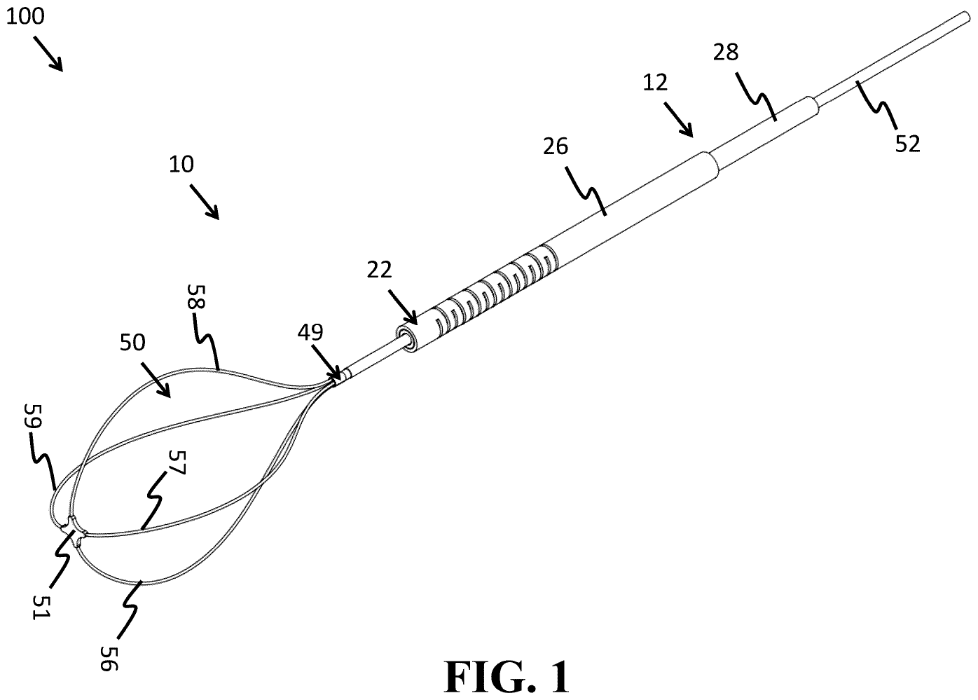

Referring now to FIG. 1, the tube assembly 10 is shown, according to some embodiments of the present disclosure. As mentioned above, the tube assembly 10 may include the steerable sheath 12, the control wire 52 disposed at least partially within the steerable sheath 12, and the basket 50 coupled to the control wire 52.

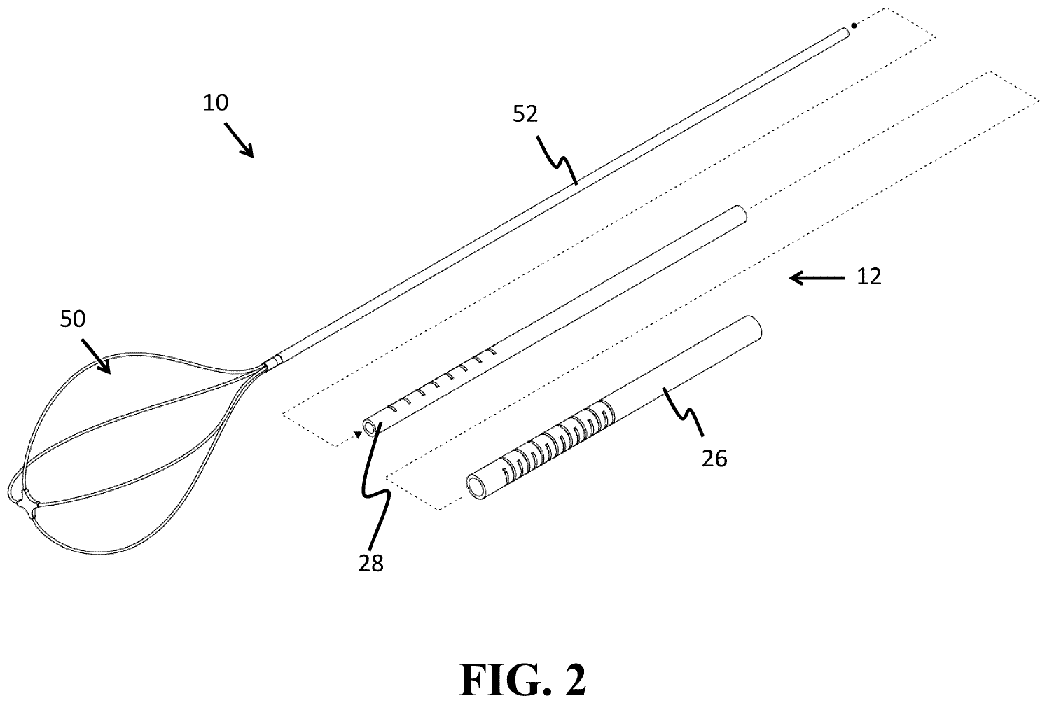

Referring now to FIG. 2, an exemplary method of providing the tube assembly 10 is shown, according to some embodiments of the present disclosure. The control wire 52 (with the basket 50 coupled thereto) may be fed through an inner lumen of the first flexible tube 28, such that the control wire 52 is disposed within the first flexible tube 28. The first flexible tube 28 (and the control wire 52 therein), in turn, may be fed through the second flexible tube 26, such that the first flexible tube 28 (and the control wire 52 therein) is disposed within the second flexible tube 26, thus providing the steerable tool 12 with the control wire 52 disposed therein.

Referring now to FIGS. 3A-3F, steerable sheath 12 is illustrated in greater detail, according to various embodiments of the present disclosure. In some embodiments, the first and second flexible tubes 28, 26 are connected or fastened to each other (e.g., via welding or adhesive bonding) at or around the distal end 22 of the steerable sheath 12 (e.g., at the distal ends of the first and second flexible tubes 28, 26). In further embodiments, the distal ends of the first and second flexible tubes 28, 26 may be joined using any suitable fastener or any suitable mode of fixation to provide a connection therebetween. The first and second flexible tubes 28, 26 may be constructed of any suitable material including, but not limited to, Nitinol, stainless steel, and plastics/polymers. In some embodiments, the first and second flexible tubes 28, 26 are formed from laser-cut hypodermic tubes.

The pre-configured state of the steerable sheath 12 may be the straight tubular configuration depicted with particular reference to FIG. 3B. For instance, the steerable sheath 12 as shown may be provided by positioning the first flexible tube 28 relative to the second flexible tube 26, such that the first flexible tube 28 is concentrically nested within, and at least partially axially aligned with, the second flexible tube 26. The steerable sheath 12 may be configured to be actuable to bend in opposite directions, as shown with reference to FIGS. 3C-3F. In other words, the steerable sheath 12 maybe configured to form a bend on one plane. Alternatively, in other embodiments, the steerable sheath 12 may be configured to form a bend along a three-dimensional curvilinear arc.

Configuring the first and second flexible tubes 28, 26 to employ the aforementioned concentric agonist-antagonist actuation scheme may be achieved by creating a deflectable portion (e.g., creating a flexible portion, selectively weaking a portion, etc.) of each of the first and second flexible tubes 28, 26 to provide respective regions of relatively low-stiffness (e.g., flexible “spines” of material) thereon. Accordingly, the first flexible tube 28 may include a first deflectable portion 40 and a first tubular sidewall portion 38 extending between a distal end 42 and a proximal end 44. Similarly, the second flexible tube 26 may include a second deflectable portion 30 and a second tubular sidewall portion 32, creating a flexible portion of the second flexible tube 26 between a distal end 34 and a proximal end 36. In some embodiments, particularly in accordance with the aforementioned agonist-antagonist actuation scheme, the first and second flexible tubes 28, 26 are joined at a location distal to the first and second deflectable portions 40, 30. For instance, the first and second tubes 28, 26 may be secured to one another at a location that is between a distal tip of the steerable sheath 12 (e.g., a tip of the distal end 22 thereof) and the first and second deflectable portions 40, 30.

In order to provide the first and second deflectable portions 40, 30, each of the first and second flexible tubes 28, 26 may have material removed (e.g., via laser cutting), thereby creating a flexible “spine” of sidewall material on each of the first and second flexible tubes 28, 26. Owing to this configuration, the first and second flexible tubes 28, 26 are axially stiff along the first and second tubular sidewall portions 38, 22 (respectively) and axially compliant along the first and second deflectable portions 40, 30 (respectively). The first and second deflectable portions 40, 30 may be configured such that the first and second flexible tubes 28, 26 feature an asymmetric elasticity (e.g., a first elasticity that is less than a second elasticity) between them. This asymmetric elasticity makes the first and second flexible tubes 28, 26 bendable (as a result of actuation) along the first and second tubular sidewall portions 38, 32 (respectively).

In some embodiments, the first and second deflectable portions 40, 30 are provided by laser-machining a slit pattern in their respective sidewalls along a portion of the length of the steerable sheath 12 that is proximal relative to the distal end 22 of the steerable sheath 12. In other words, the first and second flexible tubes 28, 26 may have a first series of cutout sections and a second series of cutout sections (respectively) spaced lengthwise along the first and second deflectable portions 40, 30 (respectively) thereby forming respective spines of flexible sidewall material on each of the first and second flexible tubes 28, 26. In the example configuration of FIGS. 3A-3D, the slit patterns on the first and second flexibles 28, 23 are formed with rectangular notches removed from the sidewalls of the tubes. The notches leave intact the first tubular sidewall portion 38 of the first flexible tube 28 and the second tubular sidewall portion 32 of the second flexible tube 26. Accordingly, the first and second deflectable portions 40, 30 may each include a series of cutout sections spaced along the longitudinal axis 92 of the steerable tool 12.

In other embodiments, and as shown with particular reference to FIGS. 3E and 3F, the first and second deflectable portions 40, 30 are provided by laser-machining a serpentine profile into their respective sidewalls along a portion of the length of the steerable sheath 12 that is proximal relative to the distal end 22 of the steerable sheath 12. Accordingly, the first and second deflectable portions 40, 30 may each include a serpentine profile of material removed from the first and second flexible tubes 28, 26 (respectively).

In some embodiments, the first and second flexible tubes 28, 26 are fastened to one another (e.g., at the distal ends 42, 34) such that the first and second deflectable portions 40, 30 are angularly oriented, relative to a longitudinal axis 38 (shown with particular reference to FIGS. 3C and 3D) of the steerable sheath 12, in directions that are offset from each other by an angle equal to or less than one-hundred and eighty degrees. For instance, the first and second deflectable portions 40, 30 may face in radially opposite directions with respect to the longitudinal axis 92 of the steerable sheath 12. In further embodiments, the relative angular orientation between first and second deflectable portions 40, 30 about the longitudinal axis 92 is between one-hundred and eighty degrees and zero degrees. In some embodiments, the relative angular orientation between first and second deflectable portions 40, 30 is one-hundred and eighty degrees. In further embodiments, the relative angular orientation between first and second deflectable portions 30, 30 about the longitudinal axis 92 is adjustable to any desired relative angular orientation to achieve an optimal bending profile of the distal end 22 of the steerable sheath 12. Accordingly, the first and second deflectable portions 40, 30 may be opposed to each other.

Because the first and second flexible tubes 28, 26 are connected at the distal end 34 of the second flexible tube 26 and the distal end 42 of the first flexible tube 28 (thus forming a connection at the distal end 22 of the assembled steerable sheath 12), the steerable sheath 12 can be actuated to cause or effectuate bending along its length through applying an axial force (e.g., an axial push and/or pull) on the first flexible tube 28 and/or the second flexible tube 26, or both of the first and second flexible tubes 28, 26 sequentially or simultaneously. For instance, by translating the proximal ends 44, 36 of the first and/or second flexible tubes 28, 26 (respectively) with respect to each other, a bending motion is produced at the distal end 22 of the steerable sheath 12 along a plane or arc of bending as defined by the first and second deflectable portions 40, 30, thereby steering the distal end 22 of the steerable sheath 12 in a bi-directional fashion. Accordingly, the steerable sheath 12 may be actuable to form a bend by axially translating the first flexible tube 28 relative to the second flexible tube 26 (or vice-versa). In other words, the steerable sheath 12 may be actuable to form a bend by relative axial translation between the first and second flexible tubes 28, 26. As discussed in greater detail below, the direction in which the steerable sheath 12 bends may depend on the push/pull directions of the actuation force exerted on the first and/or second flexible tubes 28, 26.

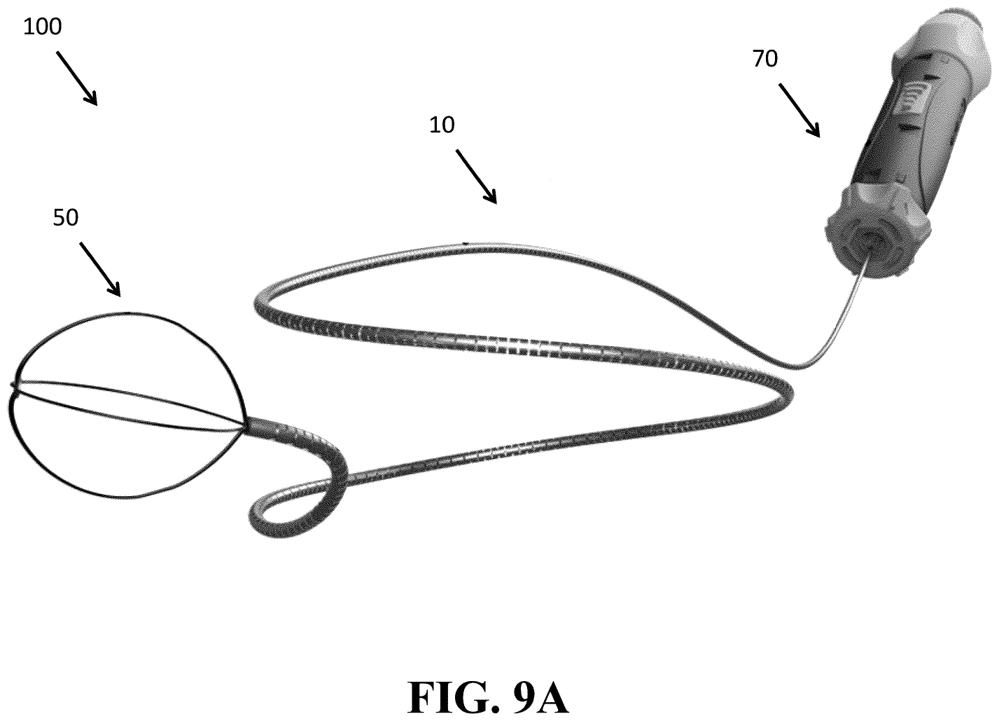

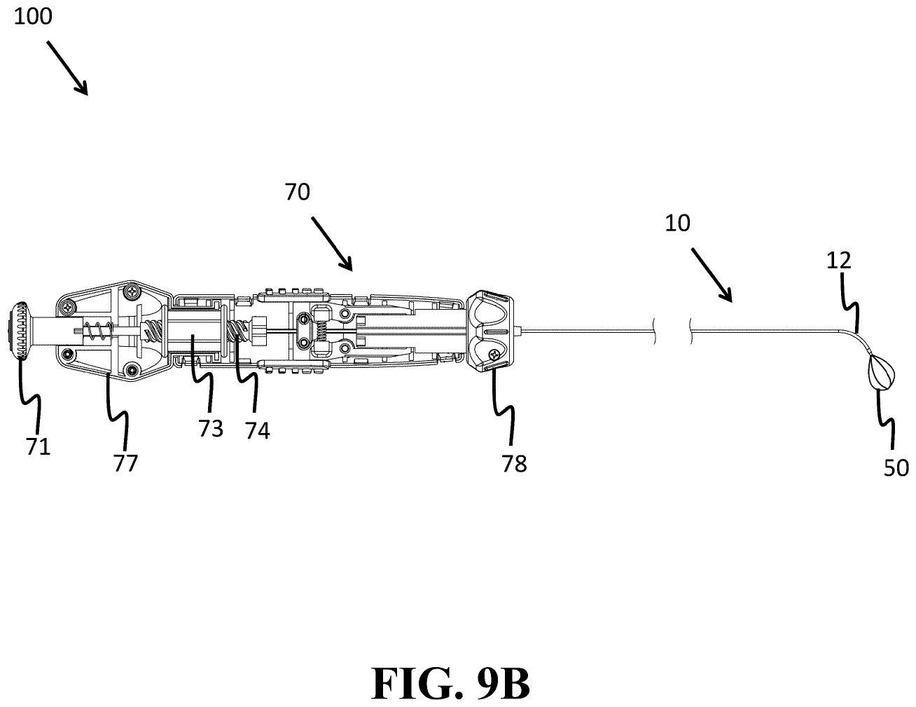

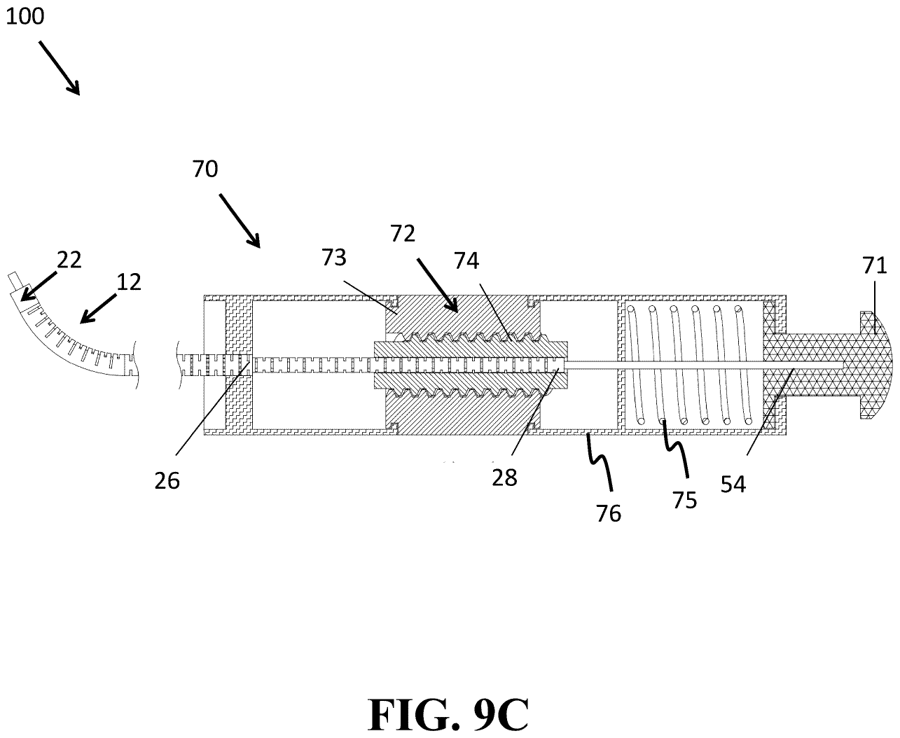

Referring particularly to FIGS. 3C and 3D, the push/pull directions along which the actuation force is applied are described with reference to the user. In particular, the actuation may be provided via a user interface 70, as described in greater detail below with reference to FIGS. 9A-13H. Actuation force applied in the “push” direction is therefore applied along the longitudinal axis 92 away from the user (e.g., toward the distal end 22 of the steerable sheath 12). Actuation force applied in the “pull” direction is therefore applied along the longitudinal axis 92 toward from the user (e.g., away from the distal end 22 of the steerable sheath 12). Following this convention, in FIG. 3C, a push force 93 is applied to the first flexible tube 28 (at its proximal end 44, for example) and a pull force 91 is applied to the second flexible tube 26 (at its proximal end 36, for example). Similarly, in FIG. 3D, a pull force 97 is applied to the first flexible tube 28 and a push force 95 is applied to the second flexible tube 26.

In some embodiments, and as generally depicted with reference to FIGS. 3C and 3D, the push/pull force applied to the first and second flexible tubes 28, 26 can be realized through the application of axial force on both tubes simultaneously. In other embodiments, and as depicted with reference to FIGS. 3E and 3F, the push/pull force applied to the first and second flexible tubes 28, 26 is relative and, therefore, can be realized through the application of axial force on one tube only. Therefore, the actuation forces applied to the proximal end 44 of the first flexible tube 28 and the proximal end 36 of the second flexible tube 26 as identified in FIGS. 3C and 3D can be realized through one of the identified push/pull force applications.

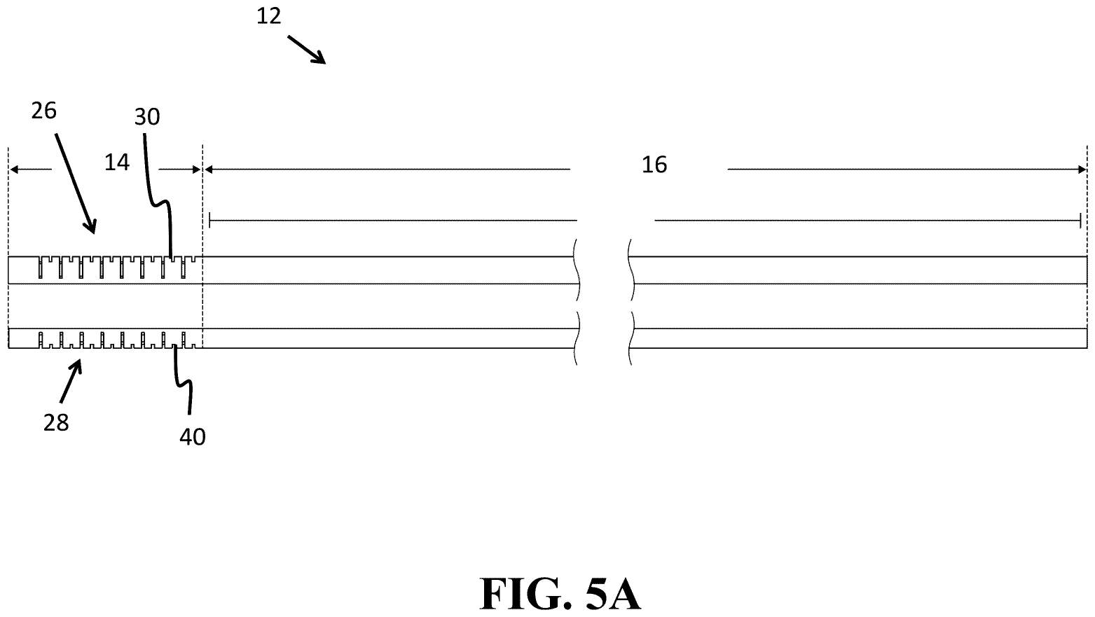

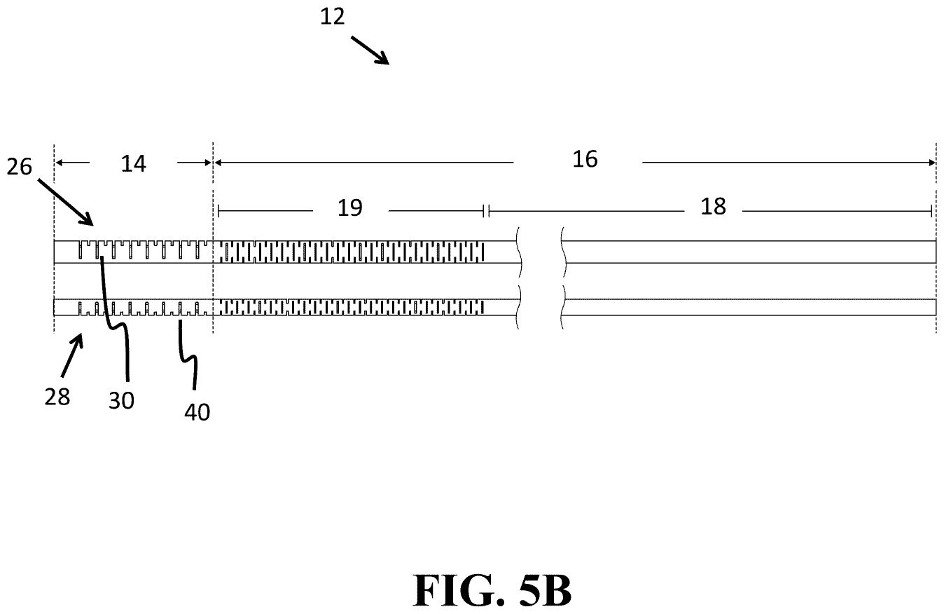

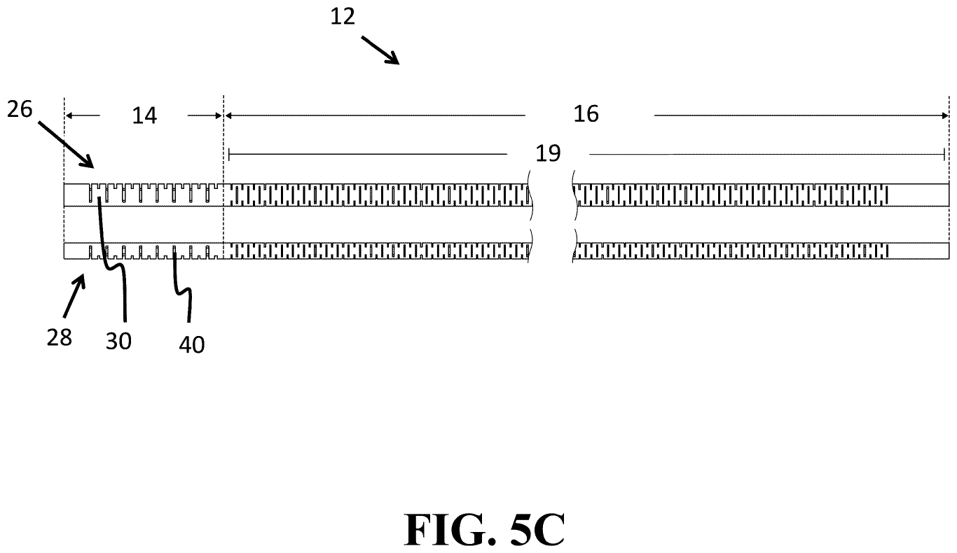

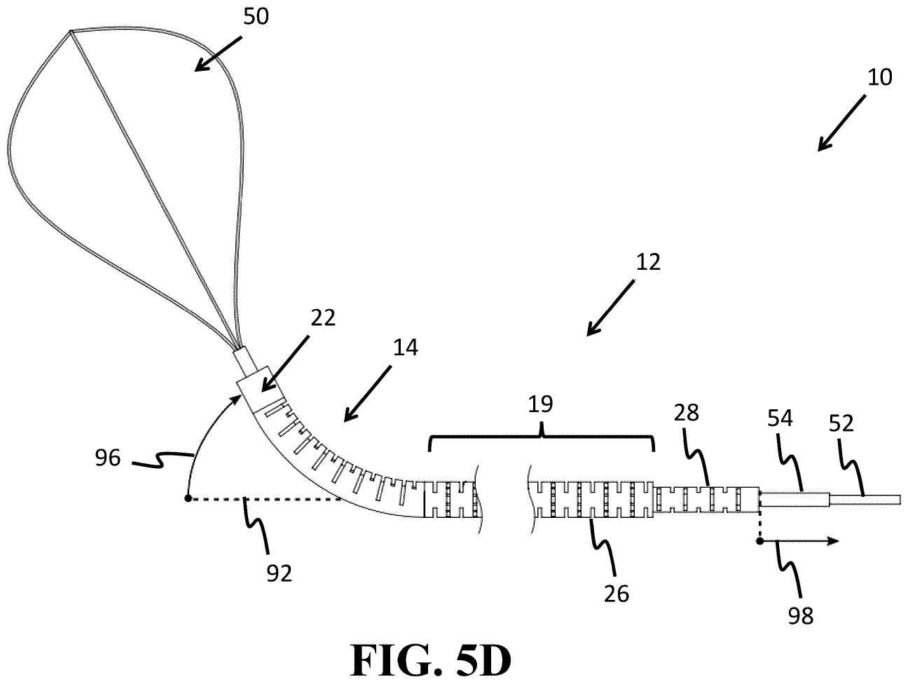

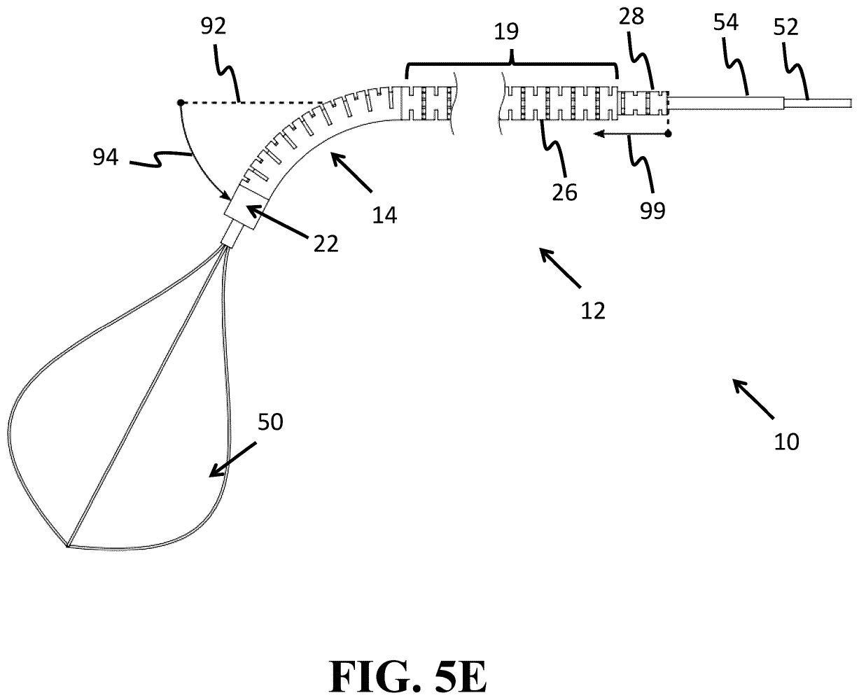

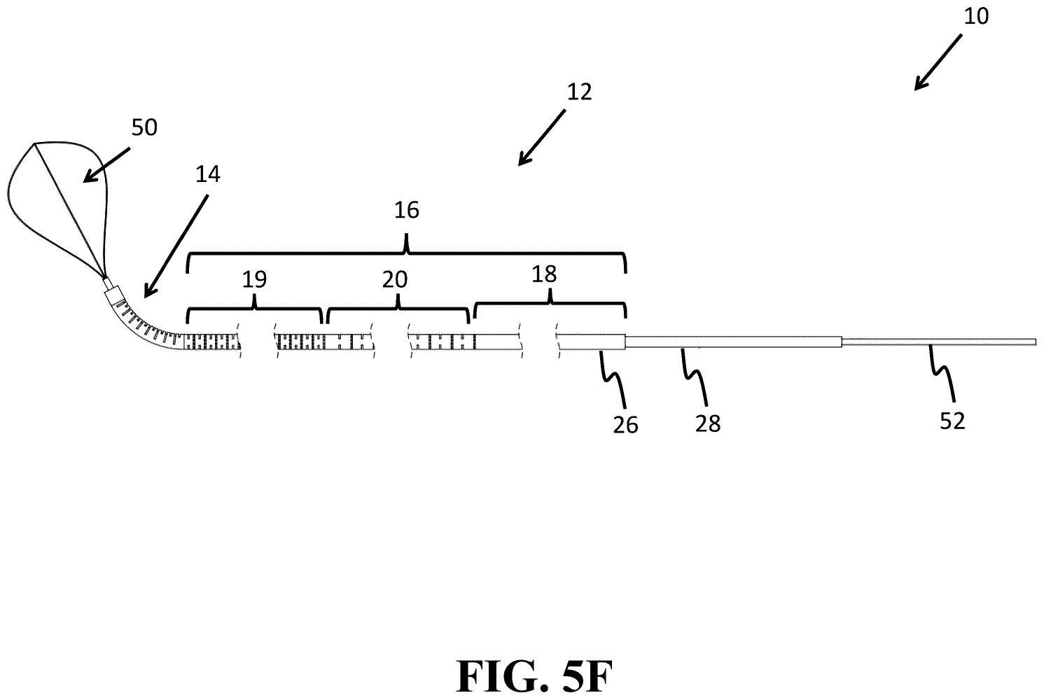

As a first example, and as depicted with particular reference to FIG. 3E with additional reference to FIG. 5D, the steerable sheath 12 can be actuated to form a bend such that the distal end 22 is steered along a path 96 by applying the pull force 97 to the proximal end 44 of the first flexible tube 28 in a direction 98 (shown with reference to FIG. 5D) while maintaining the second flexible tube 26 in a fixed axial position. Alternatively, such motion may be accomplished by applying the push force 95 to the proximal end 36 of the second flexible tube 26 in a direction 99 while maintaining the first flexible tube 28 in a fixed axial position.

As a second example, and as depicted with additional reference to FIG. 3F with additional reference to FIG. 5E, the steerable sheath 12 can be actuated to form a bend such that the distal end 22 is steered along a path 94 by applying the push force 93 to the proximal end 44 of the first flexible tube 28 in a direction 99 (shown with reference to FIG. 5E) while maintaining the second flexible tube 26 in a fixed axial position. Alternatively, such motion may be accomplished by applying the pull force 91 to the proximal end 36 of the second flexible tube 26 while maintaining the first flexible tube 28 in a fixed axial position.

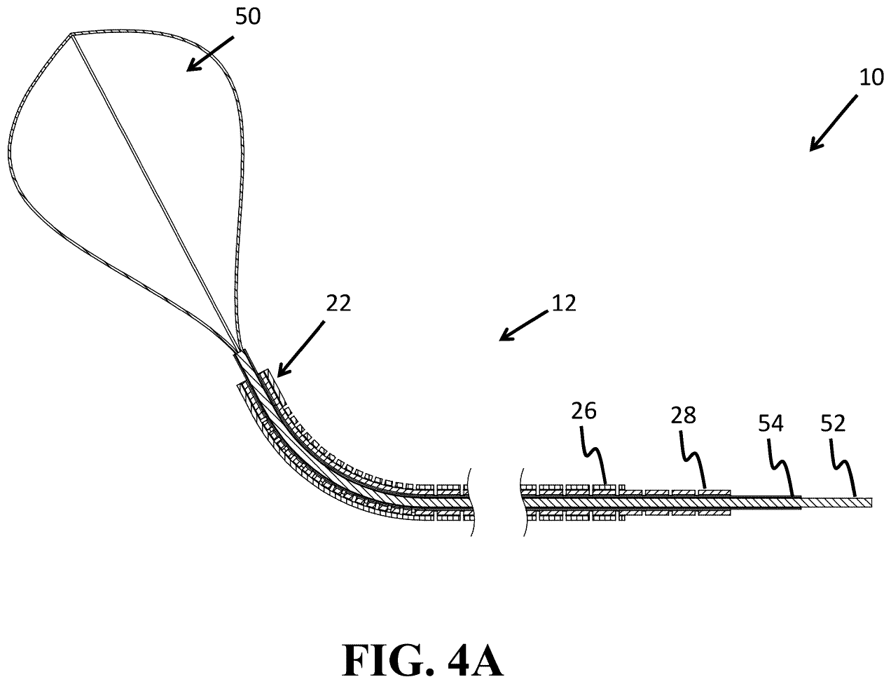

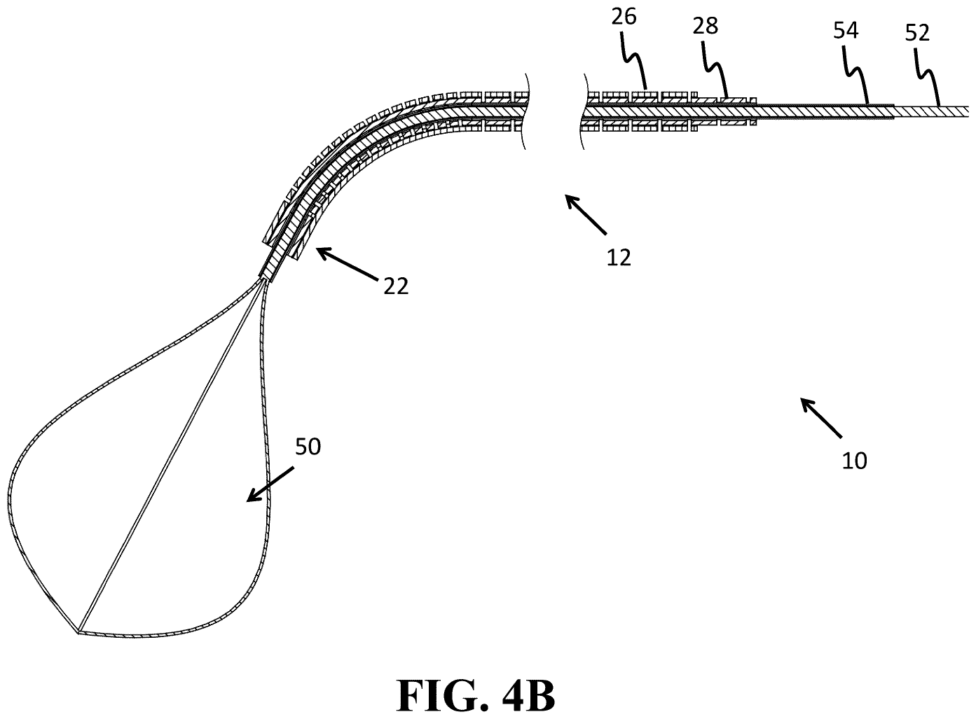

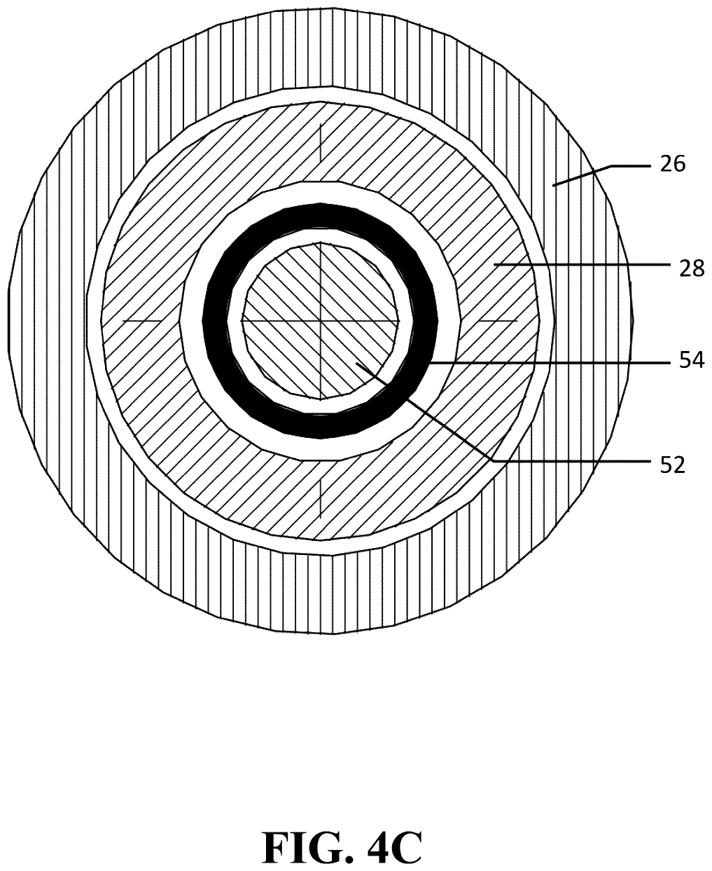

Referring now to FIGS. 4A-4C, cross sectional views of the tube assembly 10 is shown, according to some embodiments of the present disclosure. For instance, FIGS. 4A and 4B depict side-cross sectional views, and FIG. 4C depicts an axial cross-sectional view. As suggested above, at least a portion of the control wire 52 may be disposed within the steerable sheath 12. Accordingly, the control wire 52 may be disposed within the first flexible tube 28, which may be disposed within the second flexible tube 23. Each of the control wire 52, first flexible tube 28, and second flexible tube 26 may be axially movable relative to one another.

In some embodiments, the tube assembly 10 further includes a control wire liner (e.g., a tube, conduit, pipe, duct, etc.) 54 disposed about the control wire 52. Thus, the control wire liner 54 may be disposed between the control wire 52 and the first flexible tube 28. As suggested above, the first flexible tube 28 may be disposed within the second flexible tube 28. The control wire liner 54 may provide a lubricous liner layer in between the first flexible tube 28 and a control wire 52, which is disposed concentrically within the basket sheath 52. Thus, the control wire 52 may be partially positioned within the lumen of the steerable sheath 12 and movable therein along an axis of the lumen of the steerable sheath 12. In order to perform stone retrieval as described herein, the first flexible tube 28 and the second flexible tube 26 may each have a wall thickness between about fifty micrometers and about one-hundred and twenty-five micrometers.

Referring now to FIGS. 5A-5F, the steerable sheath 12 may include two or more sections, according to some embodiments of the present disclosure. For example, the steerable sheath 12 may include a steerable section 14 on a distal region of the steerable sheath 12, which may include the first and second deflectable portions 40, 30 of the first and second flexible tubes 28, 26. Accordingly, the steerable section 14 may be controlled by the user as described above. In some embodiments, the steerable sheath 12 may further include a transmission section 16 located distal to the steerable section 14. Accordingly, the steerable sheath 12 may include the steerable section 14 that is actuable to form a bend, and the transmission section 16 that is located further from the basket 50 than the steerable section 14. The transmission section 16 may be a section of the steerable sheath 12 that connects the steerable section 14 to a user interface. In some embodiments, the transmission section 16 is constructed from the same piece of tubing as the steerable section 14. In other words, the steerable and transmission sections 14, 16 may be formed as a single piece of material. In other embodiments, the transmission section 16 is a separate tube that is adhered or welded onto steerable section 14.

In some embodiments, and as depicted with particular reference to FIG. 5A for applications where the route to the anatomical region is primarily linear, the transmission section 16 may be rigid, solid tubing. In other embodiments, and as depicted with particular reference to FIGS. 5B and 5C, in applications where the route to the anatomical region is more tortuous, the transmission section 16 may include a flexible section 19 and a rigid section 18. For example, FIGS. 5D and 5E depict the flexible section 19 of the second flexible tube 26, in particular. As described in greater detail below, the flexibility of the flexible section 19 may be greater than the flexibility of the rigid section 18. As shown, the passive section 19 of the transmission section 16 may be located closer to the steerable section 14 than the rigid section 18 of the transmission section 16.

In some embodiments, the rigid section 18 may be made from solid tubing. The rigid section 18 may be proximal to both the steerable section 14 and the flexible section 19 and can be used as a rigid end on which a linear force may be applied (via the user interface 70, for instance) to extend the steerable sheath 12 through the route towards the anatomical region, as well as actuate the steerable sheath 12 to form a bend. In turn, the flexible section 19 may be flexible enough to conform to a tortuous pathway towards the anatomical region.

In some embodiments, the flexible section 19 is compliant in bending to be passively flexible in all bending directions while maintaining relatively high torsional and axial stiffness. Moreover, the flexible section 19 may be flexible enough to permit the operational range-of-motion (e.g., bending) typically required for navigation through the various anatomical structures of the patient.

In some embodiments, the aforementioned flexibility of the flexible section 19 is achieved through a fabricated pattern or a multitude of slots laser-machined into the first and second flexible tubes 28, 23 in order to reduce their stiffness. In other embodiments, the aforementioned flexibility of the flexible section 19 is achieved through the inclusion of a tube of a more flexible polymeric tubing (e.g., polyimide, PEBAX, Nylon, etc.). In other embodiments still, the aforementioned flexibility of the flexible section 19 is achieved by the same or similar methods used for providing the first and second deflectable portions 40, 30 of the first and second flexible tubes 28, 26, as described above. Depending on the implementation, one or both of the flexible section 19 and the rigid section 18 may include braid reinforcement.

Continuing to refer particularly to FIGS. 5B and 5C, the flexible section 19 may extend over a partial length of the transmission section 16 as shown with reference to FIG. 5B, or extend along a full or substantial length of the transmission section 16 as shown with reference to FIG. 5C. Depending on the implementation, the slot pattern that forms the flexible section 19 may be constant along the length of the flexible section, or it may vary along the length of the flexible section. This variation can be achieved by changing the spacing between subsequent slot sections (e.g., the pitch), or by increasing the arc length of each slot around the tube's circumference (e.g., the cut fraction). Such variation may be desirable depending on the anatomy being accessed. For example, it may be advantageous for a distal portion of the transmission section 16 to have a higher flexibility to better conform to the natural anatomy being accessed, and for a proximal section of the transmission section 16 to have a lower flexibility for better push-ability, torque-ability, and device control via the user interface 70.

Referring particularly to FIG. 5F, the transmission section 16 of the steerable sheath 12 may further include a transition section 20 located between the flexible section 19 and the rigid section 18. The transition section 20 may provide a relatively smooth transition for the variation in material properties between the flexible section 19 and the rigid section 18. For example, FIG. 5F depicts the flexible section 19, transition section 20, and rigid section 18 of the second flexible tube 26, in particular.

As suggested above, the tube assembly 10 may further include one or more layers of braided material disposed about the first flexible tube 28 and/or the second flexible tube 26 for reinforcement purposes.

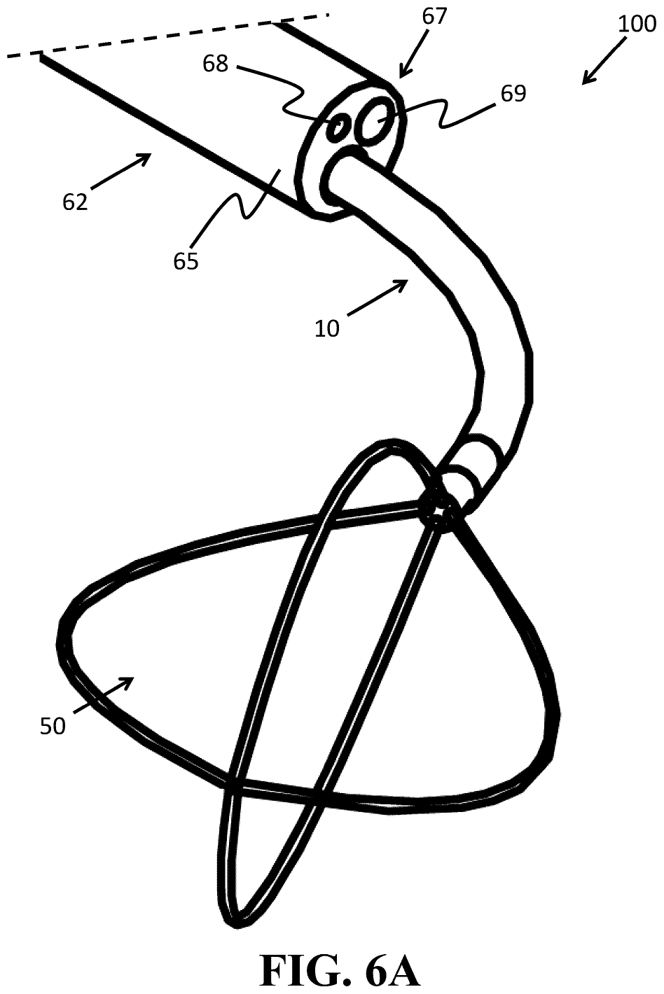

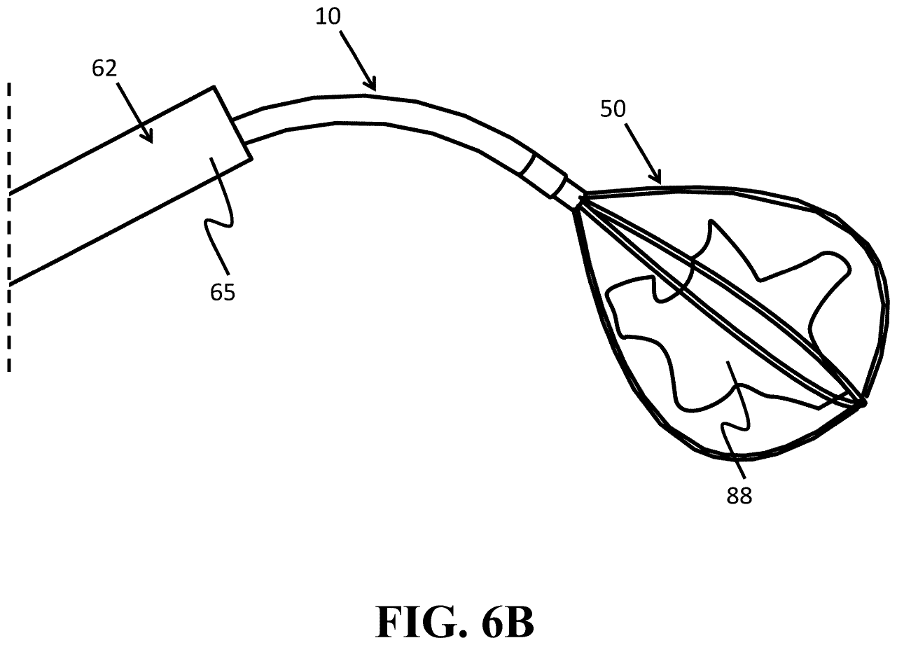

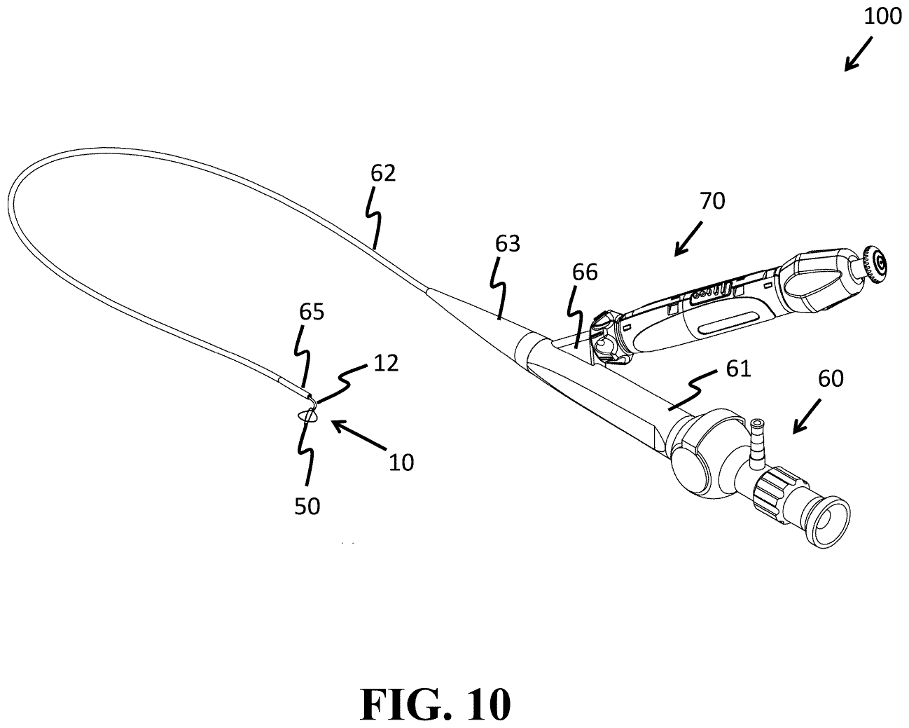

Referring now to FIGS. 6A and 6B, the apparatus 100 may include an outer tube 62, according to some embodiments of the present disclosure. For instance, the outer tube 62 may be a ureteroscope tube of a ureteroscope 60 (depicted with reference to FIGS. 10-12F). As shown, the tube assembly 10 may be disposed within the outer tube 62, and extend out of a distal end 65 of the outer tube 62. For instance, during use, the tool assembly 10 may be extended through the outer tube 62 such that the steerable sheath 12 projects out of the distal end 65. In this sense, once the steerable sheath 12 has been extended through the outer tube 62, the steerable section 14 of the steerable sheath 12 (depicted with reference to FIGS. 5A-5C) may protrude from the opening formed by the distal end 65 of the outer tube 62. The flexible section 19 proximal to the active steerable section 14, on the other hand, would be substantially disposed within the ureteroscope 62 in order to facilitate the protrusion of the active steerable section 14 through the distal end 65 as discussed above.

In some embodiments, and as shown with particular reference to FIG. 6A, the outer tube 62 further includes a tool assembly 67 located on or about the distal end 65. For example, the tool assembly 67 may include a digital image and/or video sensor 68 disposed on the distal end 65. The image and/or video sensor 68 may capture images of an area in and around the anatomical region. Depending on the implementation, the image and/or video sensor 68 may be regular camera, a thermal camera, or both. As another example, the tool assembly 67 may include one or more light sources 69 disposed on the distal end 65. The one or more light sources 69 may be LED light sources or fiber optic light sources, for example.

In some embodiments, the distal end 65 further includes an opening to facilitate the passage of other secondary tools for therapeutic purposes within the anatomical region. Accordingly, the distal tool assembly 67 may include any appropriate tools at the distal end 65 in order to perform the methods described herein. As an example, the distal tool assembly 67 may include a laser for performing laser lithotripsy on a stone 88 to break the stone 88 into smaller pieces that may be captured by the basket 50, as shown with particular reference to FIG. 6B.

Depending on the implementation, the steerable sheath 12 in its entirety may be long enough to traverse the length of the outer tube 62 within which it is disposed (e.g., between about seven-hundred and about eight-hundred and fifty millimeters, depending on the implementation), project at least a distal portion of the steerable sheath 12 out of the opening formed by the distal end 65 of the outer tube 62, and project at least a proximal portion of the steerable sheath 12 out of a proximal end of the outer tube 62 such that the aforementioned proximal portion of the steerable sheath 12 is able to interface with the one or more actuators described in greater detail below. As an example configuration, the active steerable section 14 of the steerable sheath 12 may be up to twenty-five millimeters in length, the flexible section 19 may be up to one-hundred millimeters in length, and the rigid section 18 (along with the transition section 20, where implemented) may be up to eight-hundred millimeters in length. Accordingly, the steerable sheath 12 in its entirety may be up to nine-hundred and twenty-fixe millimeters in length.

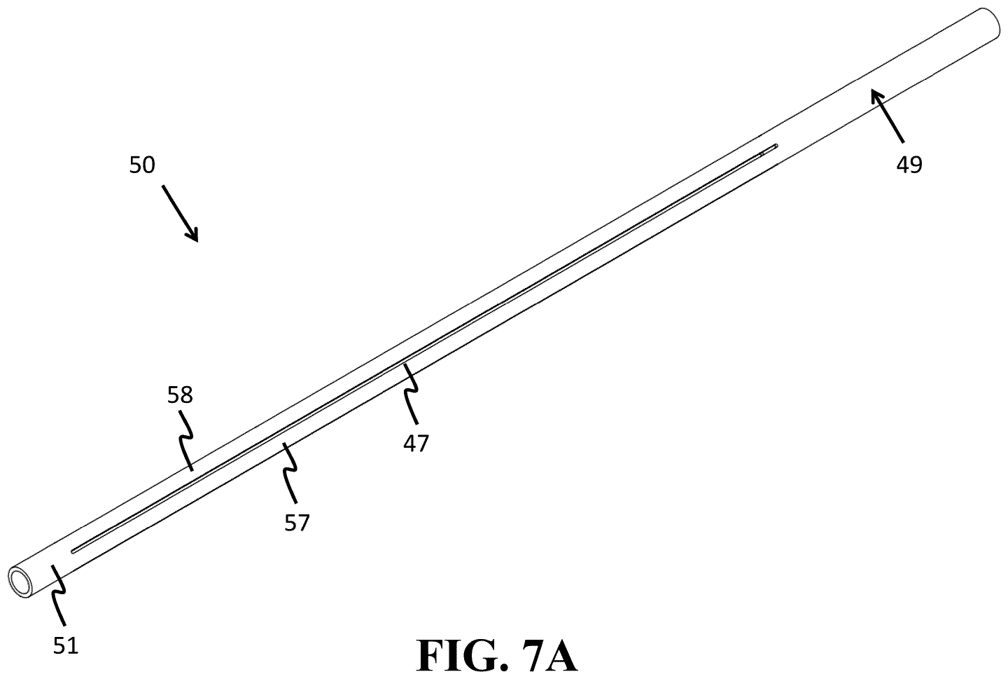

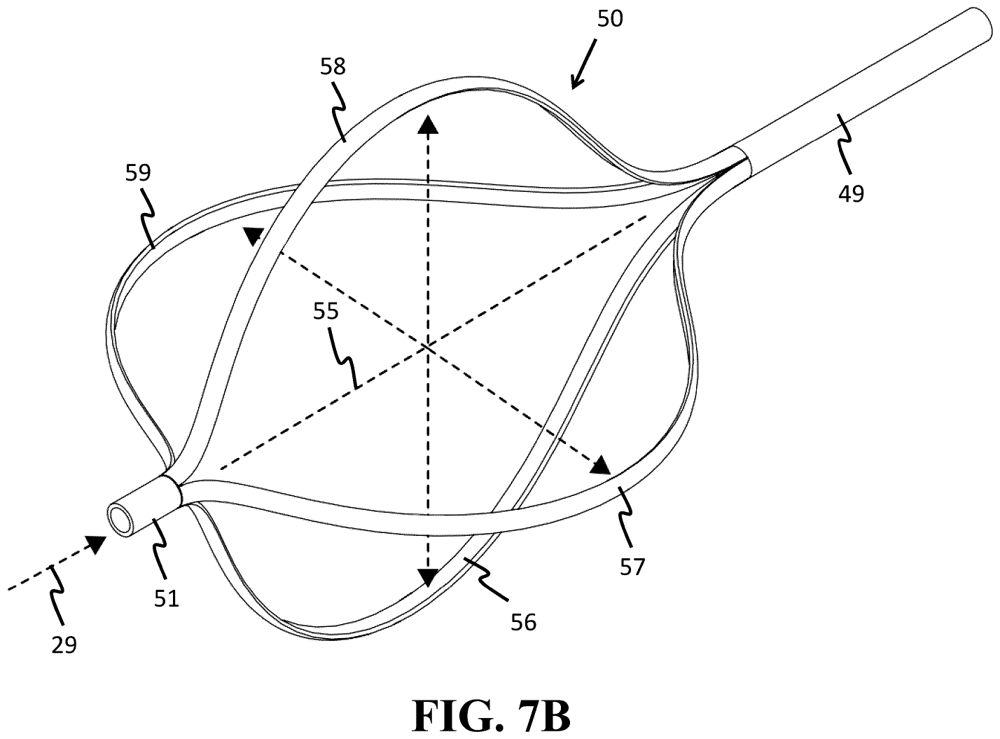

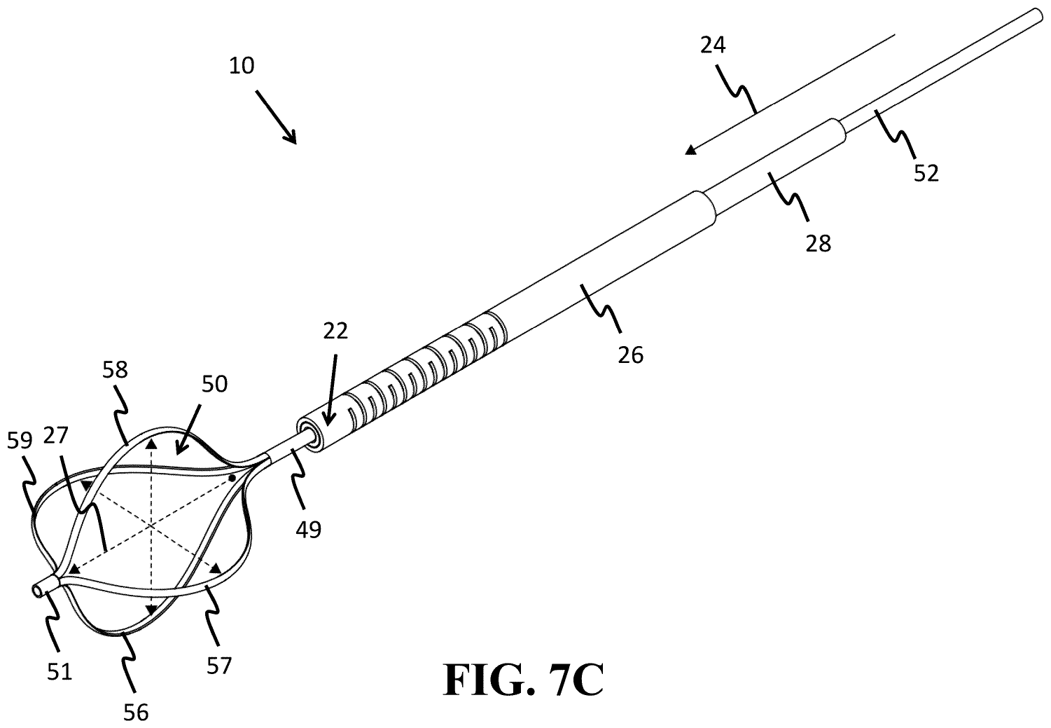

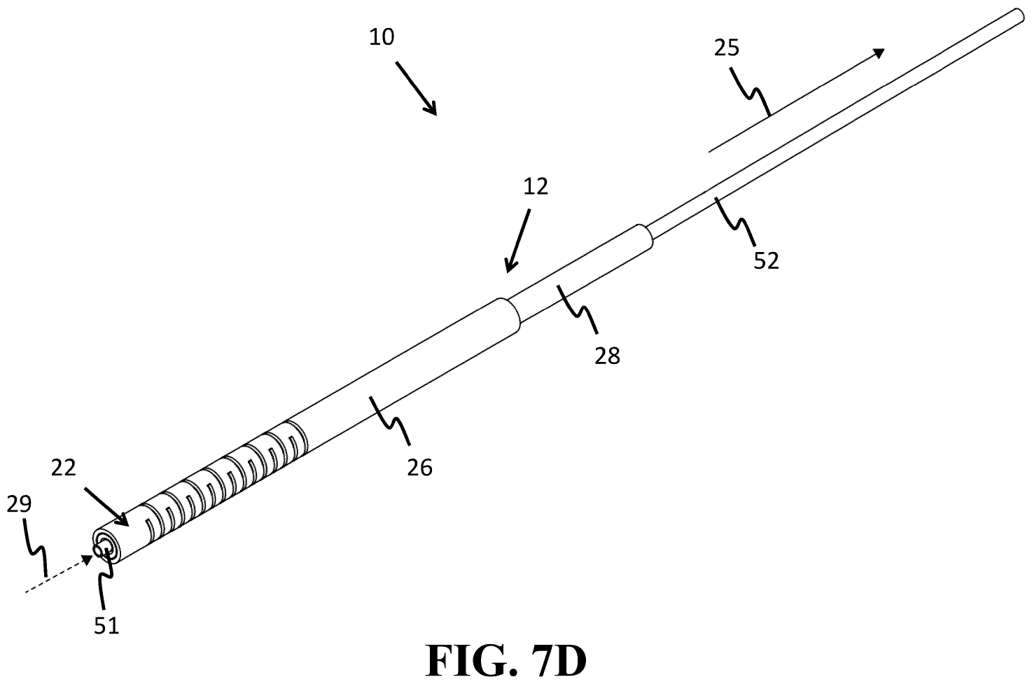

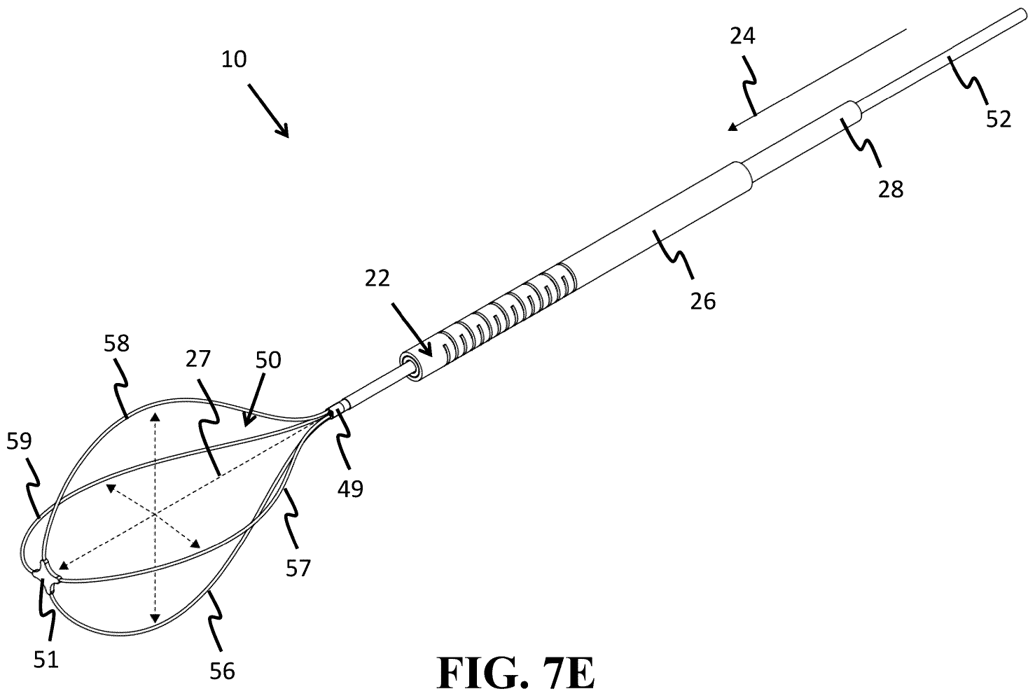

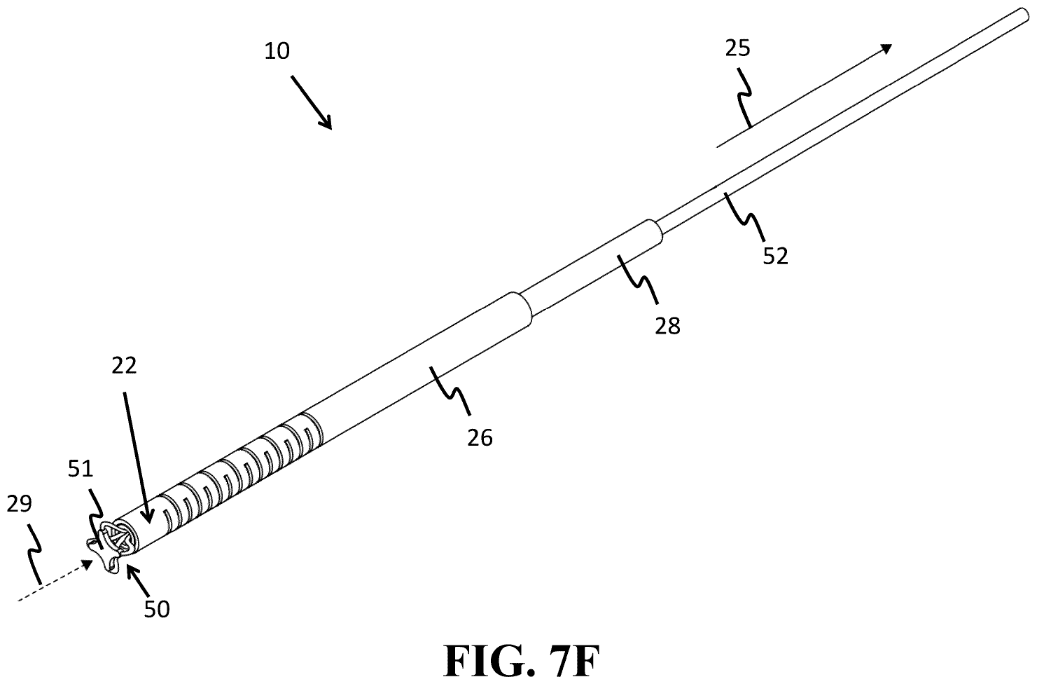

Referring now to FIG. 7A-7F, the basket 50 is shown in greater detail, according to some embodiments of the present disclosure. As mentioned above, the basket 50 may be configured to capture the stone 88 as depicted with reference to FIG. 6B. In some embodiments, the basket 50 includes a number (e.g., two, three, four, five, six, and so on) of wires each having a pre-formed curvature and attached (e.g., mechanically engaged, crimped, welded) at their distal ends to form a “cage.” For example, the basket 50 may include a first wire 56, a second wire 57, a third wire 58, and a fourth wire 59. The wires 56-59 may be made of any suitable material including, but not limited to, super-elastic Nitinol. In some embodiments, each of the wires 56-59 are coupled to each other at their distal ends, and may be coupled, at their proximal ends, to the control wire 52. For instance, the wires 56-59 may be coupled to one another at their distal ends via a basket tip 51. As a first example, and as depicted with reference to FIGS. 7A-7D, the basket tip 51 may be a cylindrical member (e.g., a laser-machined Nitinol tube) that receives, or is formed by, the distal ends of the wires 56-59. Accordingly, the wires 56-59 may be connected at their distal ends via a cylindrical member. As a second example, and as depicted with reference to FIGS. 7E and 7F, the basket tip 51 may be a substantially flat member (e.g., a polymeric joining disk) that is oriented perpendicular to the longitudinal axis 92 of the steerable sheath 12. Accordingly, the wires 56-59 may be connected at their distal ends via a joining disk. It should be appreciated that, while the basket 50 as depicted herein includes four wires, (e.g., the wires 56-59), such depictions are exemplary in nature and thus the basket 50 may include more or less wires in other embodiments of the present disclosure.

In some embodiments, the wires 56-59 unite at their proximal ends at a basket base 49, which may be coupled to the control wire 52. The control wire 52, in turn, may run the length of the steerable sheath 12 and interface with one or more actuators (which may be disposed on the actuator 70 as discussed in greater detail below) in order to be actuated by receiving pushing and pulling forces. For instance, such pushing forces may advance the control wire 52 in a direction 24 as depicted with reference to FIGS. 7C and 7E, while such pulling forces may retract the control wire 52 in a direction 25 as depicted with reference to FIGS. 7D and 7F. As discussed in greater detail below, advancing movements of the control wire 52 may cause the basket 50 to project out of the distal end 22 of the steerable sheath 12 and expand, while retreating movements of the control wire 52 may cause the basket 50 to retract into the distal end 22 of the steerable sheath 12 and contract.

In response to such pushing forces and the resulting translation of the control wire 52 in the direction 24, the basket 50 coupled thereto may be advanced in a direction 27 to project out of the distal end 22 (e.g., via an opening formed in the distal end 22) of the steerable sheath 12 and thus expand into the depicted configuration (e.g., a “released state”). Conversely, in response to such pulling forces and the resulting translation of the control wire 52 in the direction 25, the basket 50 may be retreated in a direction 29 to retract into the opening formed in the distal end 22 and thus contract into a “collapsed state” in order to capture (e.g., grab, encapsulate, take hold of, etc.) the stone 88 to be removed via an endoscopic approach. Such expansion and/or contraction may be facilitated by advancing and/or retreating movements (respectively) of the control wire 52 coupled to the basket 50 at the basket base 49. Thus, advancing movements of the control wire 52 (in response to the aforementioned pushing forces on the control wire 52) may cause the basket 50 to project out of the opening formed by the distal end 22 of the steerable sheath 12 and expand, while retreating movements of the control wire 52 (in response to the aforementioned pulling forces on the control wire 52) may cause the basket 50 to retract into the opening formed by the distal end 22 and contract.

Accordingly, based on actuation applied to the control wire 52, the basket 50 may project out of and retract into the opening formed by the distal end 22 of the steerable sheath 12. In other words, the opening formed by the distal end 22, at its inside edge, may apply an encapsulating force on the wires 56-59 of the basket 50 when the basket 50 is retracted into the opening, causing the overall dimensions of the basket 50 to contract. For example, a radial force applied on the wires 56-59 by the opening of the distal end 22 when the control wire 52 is actuated with a pulling force causes the contraction of the basket 50 into the collapsed state, which may be utilized to encapsulate the stone 88 that is positioned within the inner bounds of the basket 50. As a corollary, when the basket 50 is projected out of the opening of the distal end 22 due to a pushing force on the control wire 52, the radial force is (at least partially) removed, allowing the basket 50 to passively expand into the released state.

In some embodiments, and as depicted with particular reference to FIGS. 7A and 7B, the basket 50 is constructed from a thin walled Nitinol tube that has been laser-machined with a number of linear slots or slits (including a linear slot 47, for example) positioned axially along the length of the tube, in a section of the proximal end of the tube. As mentioned above, advancing movements of the control wire 52 (in response to the aforementioned pushing forces on the control wire 52) may cause the basket 50 to project out of the opening formed by the distal end 22 of the steerable sheath 12 and expand into the released state. For instance, advancing movements of the control wire 52 may compress the basket base 49 axially. As the basket base 49 is compressed axially, spines defined by the linear slots (e.g., the four wires 56-59) buckle outwards, expanding the basket 50 into the released state as depicted in greater detail with particular reference to FIG. 7B. In some embodiments, the basket 50 is configured to be annealed (e.g., heated past its austenitic transition temperature) while in the released state to preserve the depicted expanded shape of the basket 50. In some embodiments, particularly where the basket 50 is configured as shown, the control wire 52 is a continuation of the basket 50. In other words, the control wire 52 and the basket 50 may be provided as a single component.

In some embodiments, the number of slots machined into the tube define the number of spines that comprise the expanded cage. For instance, as depicted with particular reference to FIGS. 7A and 7B, the basket 50 includes the four wires 56-59. Accordingly, the basket 50 may include four corresponding slots (including the slot 47).

As mentioned above, retreating movements of the control wire 52 (in response to the aforementioned pulling forces on the control wire 52) may cause the basket 50 to retract into the opening formed by the distal end 22 and contract into the collapsed state. In some embodiments, and as shown with particular reference to FIG. 7D, the basket 50 may be withdrawn inside the steerable sheath 12, causing the aforementioned spines (e.g., the wires 56-59) to collapse, thereby contracting the basket 50 into the collapsed state. For instance, a diameter of the basket 50 in the collapsed state (e.g., when formed as a tube as depicted with particular reference to FIG. 7A) may be smaller than an inner diameter of the first flexible tube 28 of the steerable sheath 12, allowing the basket 50 to be fed in through the proximal end of the steerable sheath. In other embodiments, and as shown with particular reference to FIG. 7F, the basket 50 may be withdrawn inside the steerable sheath 12 until the basket tip 51 (e.g., when provided as a substantially flat member) makes contact with the distal end 22 of the steerable sheath 12.

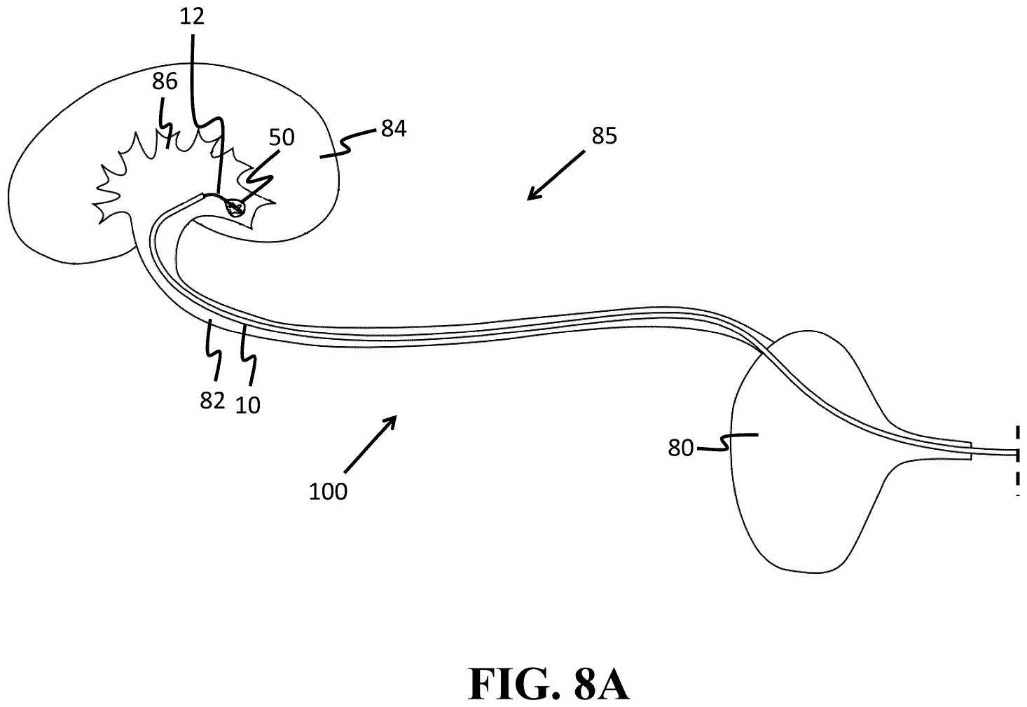

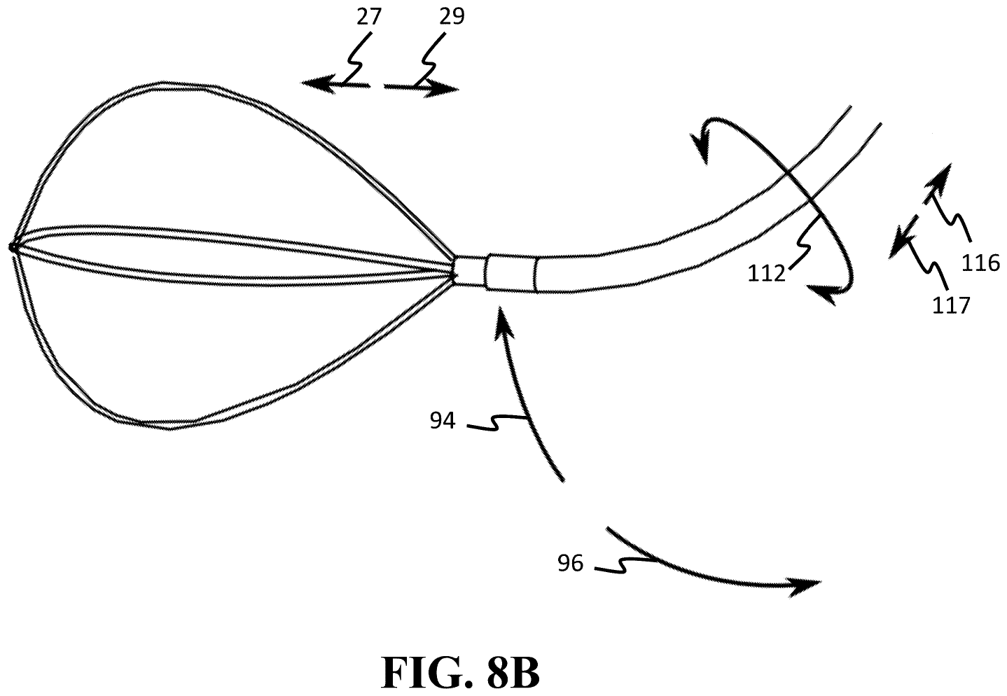

Referring now to FIGS. 8A and 8B, the apparatus 100 is shown being used to retrieve the stone 88 from within a patient's kidney 84, according to some embodiments of the present disclosure. For instance, the steerable sheath 12 may be advanced through a urinary system 85 of the patient. In particular, advancing the steerable sheath 12 through the urinary system 85 of the patient may include advancing the steerable sheath 12 through a ureter 82 of the patient and towards a calyces 86 of the patient's kidney 84, such that the distal end 22 of the steerable sheath 12 is located in or near the calyces 86. Accordingly, an exemplary method of retrieving the stone 88 may include a first step where the outer tube 62 is inserted into a patient (e.g., extended in a direction 117) such that the distal end 65 of the outer tube 62 is positioned in or near the calyces 86 of the patient. For instance, the outer tube 62 may be extended through the ureter 82 (using a bladder 80 as an entrance) and toward the calyces 86 of a patient's kidney 84, such that the distal end 65 of the outer tube 62 is located in or near the calyces 86. This exemplary method may be alternatively conducted without the outer tube 62 (and thus be accomplished solely with the steerable sheath 12). The various components of the apparatus 100, particularly the tool assembly 10, may be configured with appropriate dimensions for accessing and navigating such anatomical regions within the patient.

A second step of the exemplary method may include the tube assembly 10 is inserting the tube assembly 10 into, and extending the tube assembly 10 through, the outer tube 62, until at least a portion of the steerable sheath 12 projects out of the distal end 65. It should be appreciated that The tube assembly 10 can be extended through the outer tube 62 before, during, or after the extension of the outer tube 62 toward the calyces 86. It should further be appreciated that, in some embodiments, the apparatus 100 does not include the outer tube 62, and thus the tube assembly 10 itself may be inserted into the patient such that the distal end 22 of the steerable sheath 12 is positioned in or near the calyces 86 of the patient. Thus, the tool assembly 10 itself can be extended in the direction 117 in order to accomplish the methods discussed herein. Ultimately, and as shown, the basket 50 may be positioned within the calyces 86 and be operable to retrieve the stone 88.

A third step of the exemplary method may include advancing the basket 50 relative to the distal end 22 of the steerable sheath, such that the basket projects out of the opening formed by the distal end 22 and expands into the released state as discussed above. For instance, the third step may include advancing the control wire 52 in the direction 24, which causes the basket 50 to advance in the direction 27 and expand into the released state.

A fourth step of the exemplary method may include actuation and/or rotation of the steerable sheath 12, such that the basket 50 is navigated toward the stone 88. For instance, the steerable sheath 12 may be actuated to form a bend along the paths 94 and 96 as discussed above. Additionally, the tube assembly 10 itself may be rotated (e.g., rotation 112) about a circumferential axis defined by a proximal portion of the steerable sheath 12 that is not deflected. In other words, rotation of the steerable sheath 12 causes a circumferential translation of the basket 50.

A fifth step of the exemplary method may include retracting the basket 50 (at least partially) into the distal end 22 by retracting the control wire 52 in the direction 25 as discussed above, such that the basket is retreated in the direction 29 and contracts into a collapsed state, thus capturing the stone 88.

A sixth step of the exemplary method may include removing the tube assembly 10 (along with the basket 50 and the captured stone 88) out of the patient. In other words, the tube assembly may be withdrawn in a direction 116.

Accordingly, the present disclosure provides a method of performing endoscopic surgery including providing the steerable sheath 12, the control wire 52 disposed within the steerable sheath 12, and the basket 50 disposed on the control wire; forming a bend in the steerable sheath 12, such that the bend causes the distal end 22 of the steerable sheath 12 to be steered toward an anatomical region within the patient (which may be the kidney 84 of the patient, the calyces 86 therein, or the region of the stone 88 therein, among other regions); advancing the control wire 52 relative to the steerable sheath 12, such that advancing the control wire 52 relative to the steerable sheath 12 causes the basket 50 to project out of the distal end 22 of the steerable sheath 12 and expand, thereby positioning the basket 50 about an object (such as the stone 88) located within the aforementioned anatomical region; and retreating the control wire 52 relative to the steerable sheath 12, such that retreating the control wire 52 relative to the steerable sheath 12 causes the basket 50 to retract into the distal end 22 of the steerable sheath 12 and contract, thereby retaining the aforementioned object within the basket 50. Additionally, the aforementioned method provided herein may include advancing the steerable sheath 12 through the ureter 82 (using the bladder 80 as an entrance) and toward the calyces 86 of the patient's kidney 84, such that the distal end 65 of the outer tube 62 is located in or near the calyces 86. Thus, the basket 50 may be manipulated as discussed above in order to retain the stone 88.

Referring now to FIGS. 9A-9C, the apparatus 100 may further include the user interface 70 coupled to the tube assembly 10, according to some embodiments of the present disclosure. For instance, the user interface 70 may be a handle. Depending on the implementation, the user interface 70 includes one or more actuators, or may be manually maneuvered, in order to enable the user to impart the various degrees of freedom associated with the function of the steerable sheath 12 and/or the basket 50 located thereon (e.g., the rotation 112 of the tube assembly 10, extension and withdrawal of the tube assembly 10 in the directions 116, 117, steering of the steerable sheath 12 along the paths 94, 96, as well as movement of the control wire 52 in the directions 24, 25, thereby advancing or receding the basket 50 in the directions 27, 29, as discussed above with reference to FIG. 8B). In other words, the user interface 70 may include actuator(s) associated with: the expansion (released state) and/or contraction (collapsed state) of the basket 50; forming a bend in the steerable sheath 12, thus steering the distal end 22 and the basket 50 located thereon; the extension and/or retraction of the tube assembly 10 (e.g., the steerable sheath 12 and the basket 50 located thereon) relative to the outer tube 62; and the rotation of the tube assembly 10, which may be about an axis defined by the inner lumen of the outer tube 62 within which it is disposed.

As suggested above, expansion and/or contraction of the basket 50 may be caused by linear motion of the control wire 52 relative to the steerable sheath 12 within which it is concentrically disposed, thus causing the basket 50 to project out of and/or retract into the opening formed by the distal end 22 of the steerable sheath 12. Such linear motion of the control wire 52 relative to the steerable sheath 12 may be actuated via a basket actuator 71. In some embodiments, the basket actuator 71 is a linearly sliding member (e.g., a plunger, button, etc.) that is operated via an upstroke and a downstroke, such that the linearly sliding member is linearly adjustable along an axis parallel to an axis defined by at least a portion of the control wire 52. For example, the basket actuator 71 may be rigidly coupled to the control wire 52 and/or the control wire liner 54. During operation of the basket actuator 71, the user interface 70 may hold the steerable sheath 12 in place relative to the linear motion being imparted on the control wire 52 and/or the control wire liner 52, and thus cause the basket 50 to project out of (via the downstroke) and/or retract into (via the upstroke) the steerable sheath 12. In other embodiments, linear motion of the control wire 52 relative to the steerable sheath 12 is caused by a rotary-to-linear transmission as discussed below with respect to a steerable sheath actuator 72 of the user interface 70.

In some embodiments, the basket actuator 71 includes may a biasing member (e.g., a spring return element) 75 to bias the basket 50 into either a passively released state or a passively collapsed state. For example, the biasing member 75 may be configured to bias the basket into a passively collapsed state, due to a biasing force that acts against the operation of the basket actuator 71, thus returning the basket actuator 71 to its initial state (and returning the basket 50 from a released state to a collapsed state) once an external force is no longer being provided to the basket actuator 71 (e.g., a user is no longer pressing the basket actuator 71 into a housing 76 of the user interface 70). In other words, the biasing member 75 causes the upstroke of the linearly sliding member (e.g., the plunger) of the basket actuator 71 via the biasing force, and biases against the downstroke via the biasing force. In some embodiments, the biasing member 75 operates in the reverse direction, causing the downstroke via the biasing force, and biasing against the upstroke via the biasing force.

As suggested above, actuation of the steerable sheath 12 (thereby steering the distal end 22) may be caused by linear displacement of the steerable sheath inner tube 28 relative to the steerable sheath outer tube 26 (or vice-versa). In some embodiments, such linear displacement is caused by the steerable sheath actuator 72. The steerable sheath actuator 72 may include a rotary-to-linear transmission (e.g., a leadscrew, slider-crank, barrel cam, etc.). For example, the rotary-to-linear transmission may include a lead-nut 73 interfacing with a leadscrew 74. The lead-nut 73 may be rotated by the user of the user interface 70 (by manual rotation of a knob 71 of the user interface 70, for example), resulting in linear translation of the leadscrew 74. The leadscrew 74 may, in turn, be rigidly coupled to one of the steerable sheath outer tube 26 or the steerable sheath inner tube 28 in order to translate the position of the steerable sheath inner tube 28 relative to the steerable sheath outer tube 26 (respectively).

In some embodiments, the leadscrew 74 is rigidly coupled to the steerable sheath inner tube 28, while a portion of a housing 76 of the user interface 70 retains the position of the steerable sheath outer tube 26. Thus, rotation of the lead-nut 73 results in linear movement of the steerable sheath inner tube 28 relative to the steerable sheath outer tube 26, deflecting the steerable sheath 12 to form a bend and steer the distal end 22. The rotation of the lead-nut 73 may be effectuated by a knob 77 of the user interface 70. In other embodiments, linear displacement of the steerable sheath inner tube 28 relative to the steerable sheath outer tube 26 (or vice-versa) may be caused by a linearly sliding member as discussed above with reference to the basket actuator 71.

In some embodiments, the user interface 70 includes a mechanism to passively “lock” the linear motion of the control wire 52 relative to the steerable sheath 12 when the basket actuator 71 is not being used, and/or the linear displacement of the steerable sheath inner tube 28 relative to the steerable sheath outer tube 26 (or vice-versa) when the steerable sheath actuator 72 is not being used. Passively locking such linear motions may be accomplished via non-back-drive-ability of the basket actuator 71 and/or the steerable sheath actuator 72, a spring detent mechanism, a ratchet/pawl mechanism, a friction locking mechanism, etc.

In some embodiments, the linearly sliding member (whether configured as the basket actuator 71, the steerable sheath actuator 72, or both) includes a switch-toggle mechanism in order to operate the basket actuator 71 and/or the steerable sheath actuator 72 via two separate user engagements with linear sliding member. For example, the switch-toggle mechanism may be actuated a first time (e.g., deploying the basket 50 into a released state or forming a pre-defined bend in the steerable sheath 12) to rotate and lock a cam body and compress the biasing member 75. The switch-toggle mechanism may be actuated a second time to rotate the cam body such that it is unlocked, and the compressed biasing member 75 facilitates the linear sliding member to move the basket 50 into a collapsed state or removing the bend from the steerable sheath 12.

Referring now to FIG. 10, the apparatus 100 is shown to include the tube assembly 10, the user interface 70, and the ureteroscope 60, according to some embodiments of the present disclosure. As shown, the ureteroscope 60 may be coupled to the user interface 70 and the tube assembly 10. In some embodiments, the ureteroscope 60 includes a ureteroscope handle 61 coupled to the outer tube 62 via the ureteroscope hub 63. In some alternative embodiments, the ureteroscope 60 is instead a rigid cystoscope or nephroscope.

In some embodiments, the user interface 70 is coupled to the ureteroscope 60 via a ureteroscope working channel mount 78 of the user interface 70, which may be coupled to a working port 66 of the ureteroscope 60. Accordingly, the ureteroscope working channel mount 78 may be configured to mechanically affix and spatially locate the user interface 70 relative to the ureteroscope 60. The tube assembly 10 may then be fed through the working port 66, the ureteroscope hub 63, and through an inner lumen of the outer tube 62, projecting at least partially out of an opening formed by the distal end 65 of the outer tube 62. In turn, the basket 50 may be delivered through the ureteroscope 60, and project from the distal end 22 of the steerable sheath 12 in order to perform the methods described herein.

In some embodiments, the user interface 70 includes a mechanism for rigid coupling to the working port 66 through which the tube assembly 10 is passed. For example, such a mechanism may be a female luer connector that is screwed onto a male luer fitting formed on the working port 66. As another example, the user interface 70 itself may be free-floating and feature a strain relieving member to limit the strain experienced by the basket 50 as it exits the user interface 70. As shown, the user interface 70 may be coupled to the working port 66 the ureteroscope 60.

In some embodiments, the outer diameter of the steerable sheath 12 in its entirety (e.g., the outer diameter of the second flexible tube 26 or the outer diameter of the outermost component disposed about the second flexible tube 26) may be less than the working port 66 (e.g., one and one-fifth millimeters). However, in order to permit adequate fluid flow through the outer tube 62, as may be required during typical operation of the ureteroscope 60, the outer diameter of the steerable sheath 12 in its entirety may be equal to or less than about one millimeter.

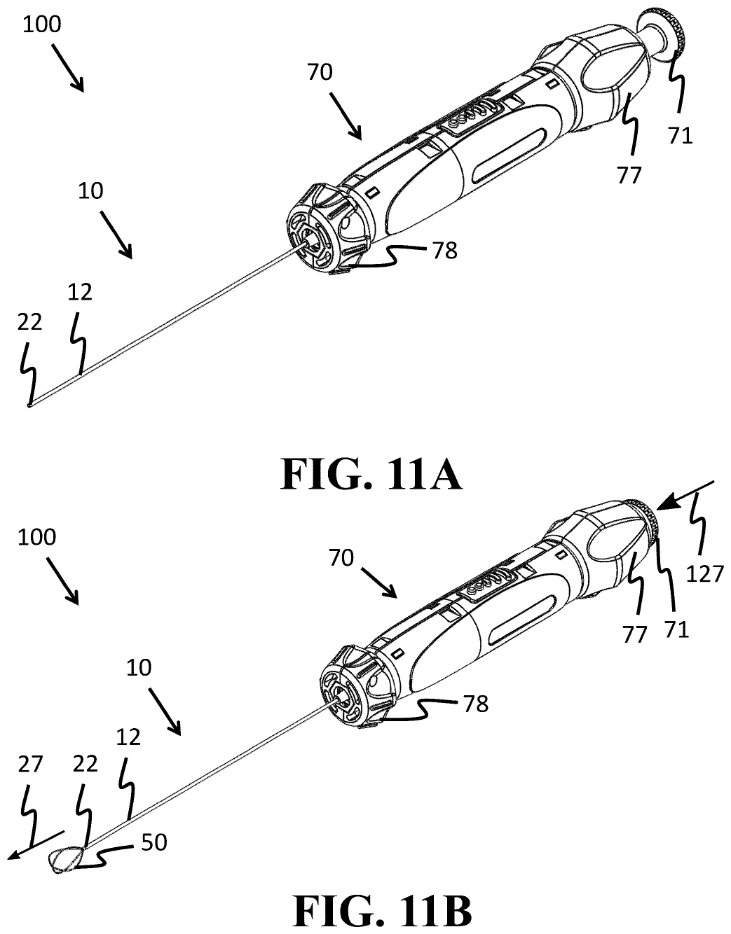

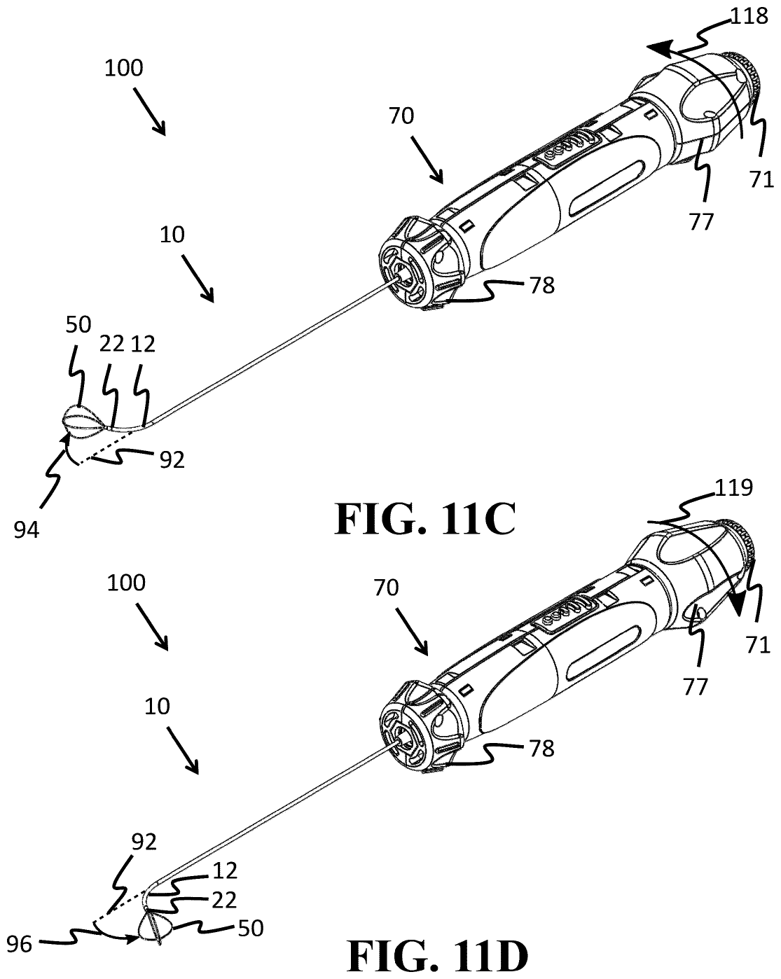

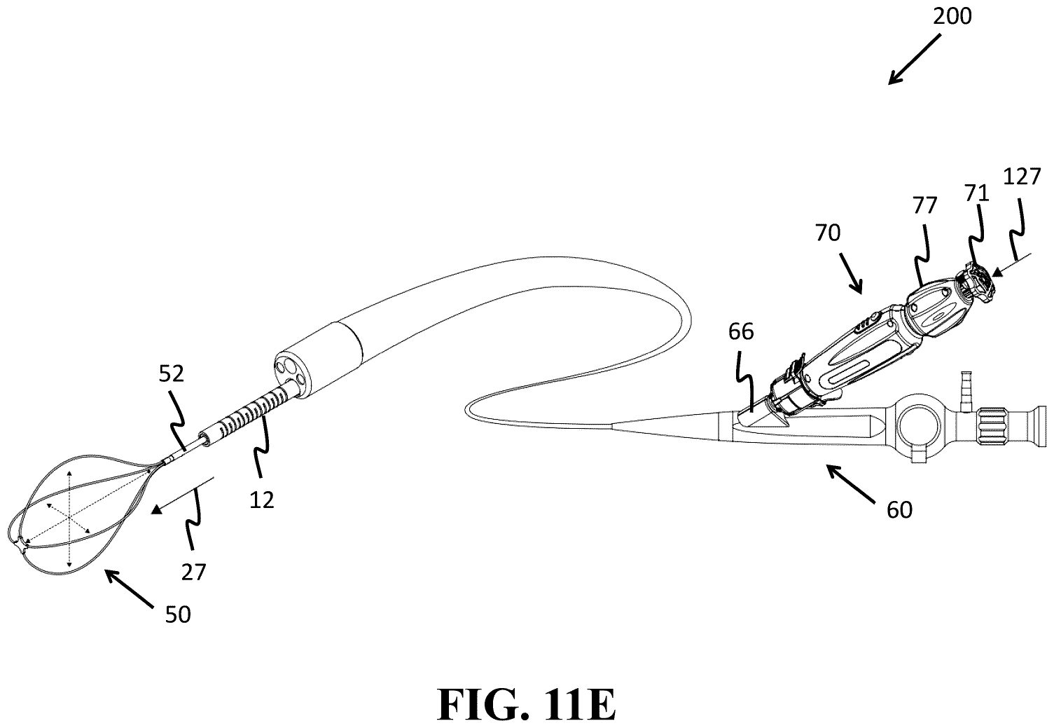

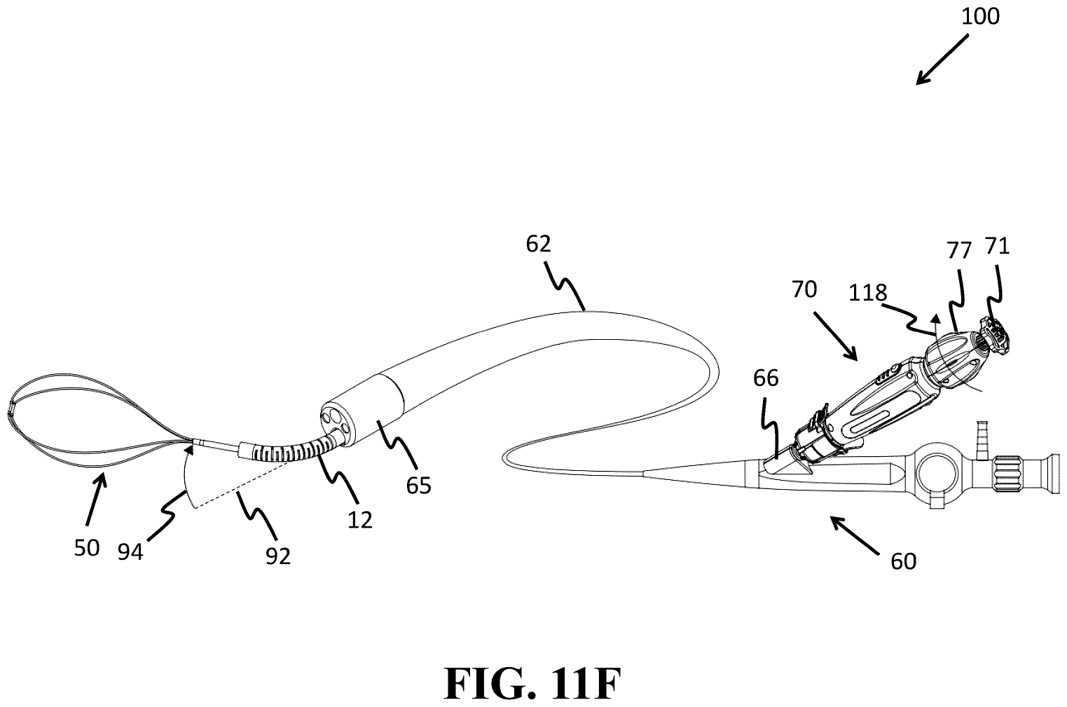

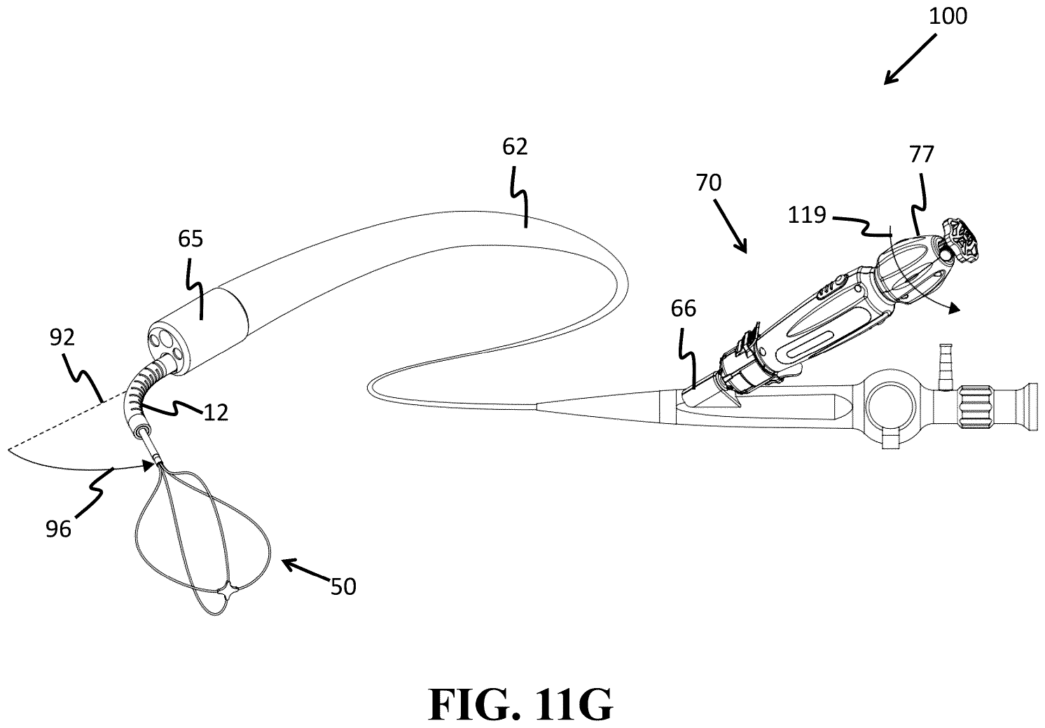

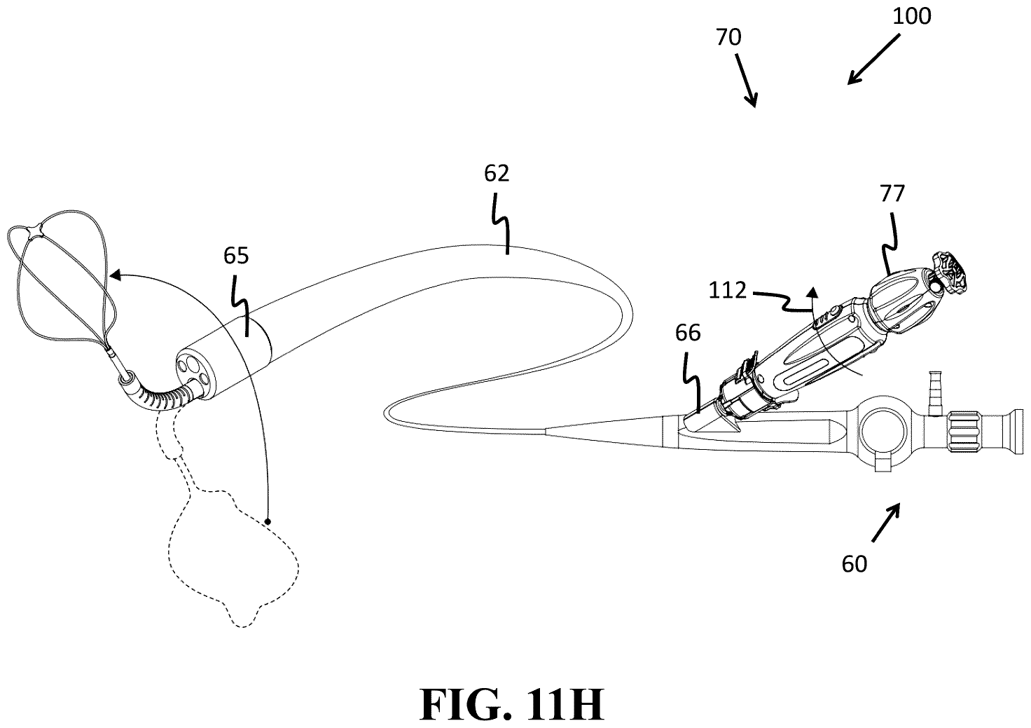

Referring now to FIGS. 11A-11H, an example implementation of the basket actuator 71 and the steerable sheath actuator 72 of the user interface 70 is shown, according to some embodiments of the present disclosure. FIGS. 11A-11D depict the apparatus 100 as operated via the user interface 70 coupled to the tube assembly 10, while FIGS. 11E-11H depict the apparatus 100 as similarly operated in conjunction with the ureteroscope 60. FIG. 11A shows a pre-configured state of the apparatus 100, where the basket 50 has yet to be projected through the distal end 22 of the steerable sheath 12. FIGS. 11B and 11E show the basket actuator 71 being actuated with a force in a direction 127, resulting in the basket 50 being advanced in the direction 27 and projecting out of the distal end 22 of the steerable sheath 12 to form the released state of the basket 50. FIGS. 11C and 11F show the knob 71 of the user interface 70 being rotated in a first direction 118, resulting in the actuation of the steerable sheath actuator 72 to form a bend in the steerable sheath 12, thus steering the basket 50 on the distal end 22 of the steerable sheath 12 along the path 94 relative to the longitudinal axis 92. FIGS. 11D and 11G show the knob 71 being rotated in a second direction 119, resulting in actuation of the steerable sheath actuator 72 to form a bend in the steerable sheath 12, thus steering the steering the basket 50 on the distal end 22 of the steerable sheath 12 along the path 96 relative to the longitudinal axis 92. FIG. 11H shows the user interface 70 being rotated rotated (e.g., rotation 112) about a circumferential axis defined by a proximal portion of the steerable sheath 12 that is not deflected, thus effectuating circumferential translation of the basket 50.

In some embodiments, insertion and retraction of the tube assembly 10 is a linear translation of the tube assembly 10 (e.g., movement of the tube assembly in the directions 116 or 117 as depicted with reference to FIG. 8B), which may be relative to the outer tube 62. Such linear translation may be accomplished via moving the user interface 70 relative to a working port 66 on a ureteroscope hub 63 of the ureteroscope 60 (as discussed in greater detail below with reference to FIGS. 10-13D). For example, the user interface 70 may be coupled to the working port 66 via a linear bearing that allows the user interface 70 to maintain a coupling to the ureteroscope 60 while having a linear range of motion that allows a linear translation of the user interface 70 and the tube assembly 10 coupled thereto. In this example, the linear bearing may include a locking mechanism to maintain the translation of the user interface 70 with respect to the ureteroscope 60 when the force (e.g., manual operation) of the user interface 70 is removed. The linear bearing may further include a biasing member to bias the user interface 70 into a particular position relative to the ureteroscope 60, allowing for a passively-inserted state or a passively-retracted state of the tube assembly 10 relative to the outer tube 62. As another example, the user interface 70 may not be coupled to the working port 66. Rather, the tube assembly 10 may be inserted into the working port 66, and fed through the outer tube 62 by moving the user interface 70 relative to the working port 66.

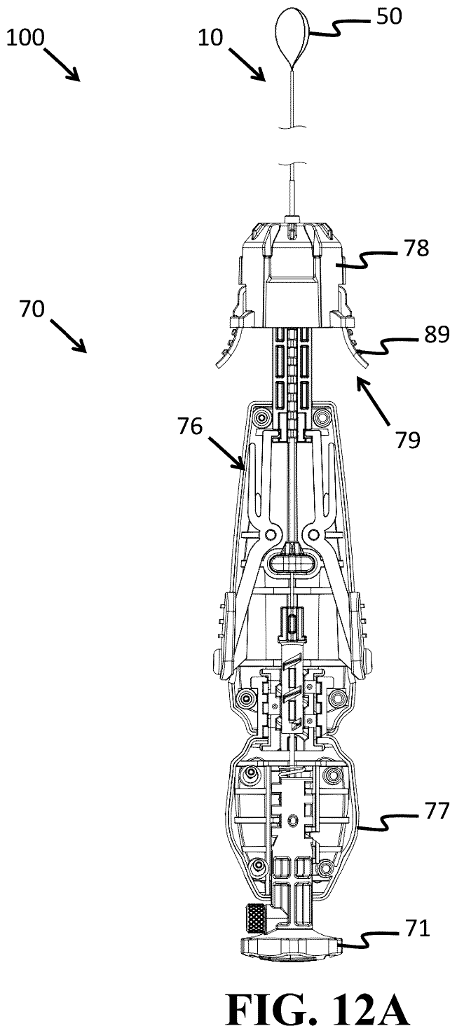

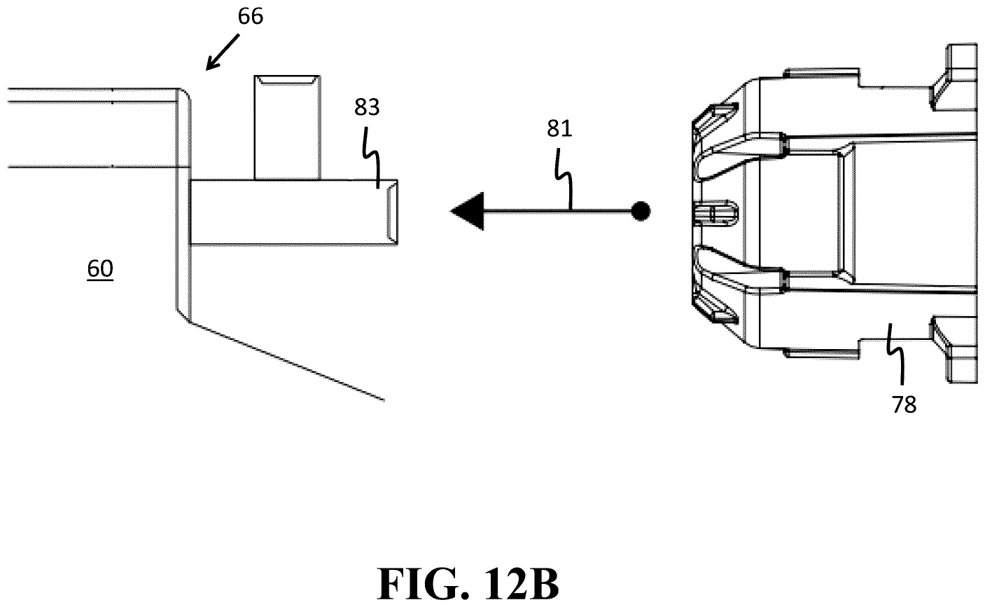

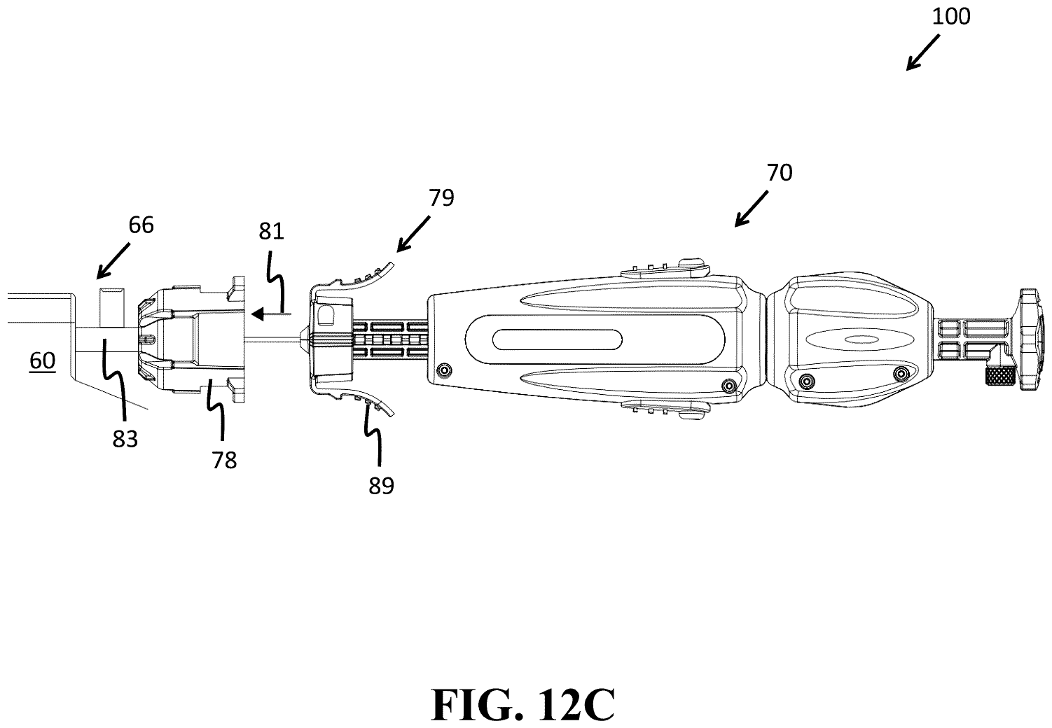

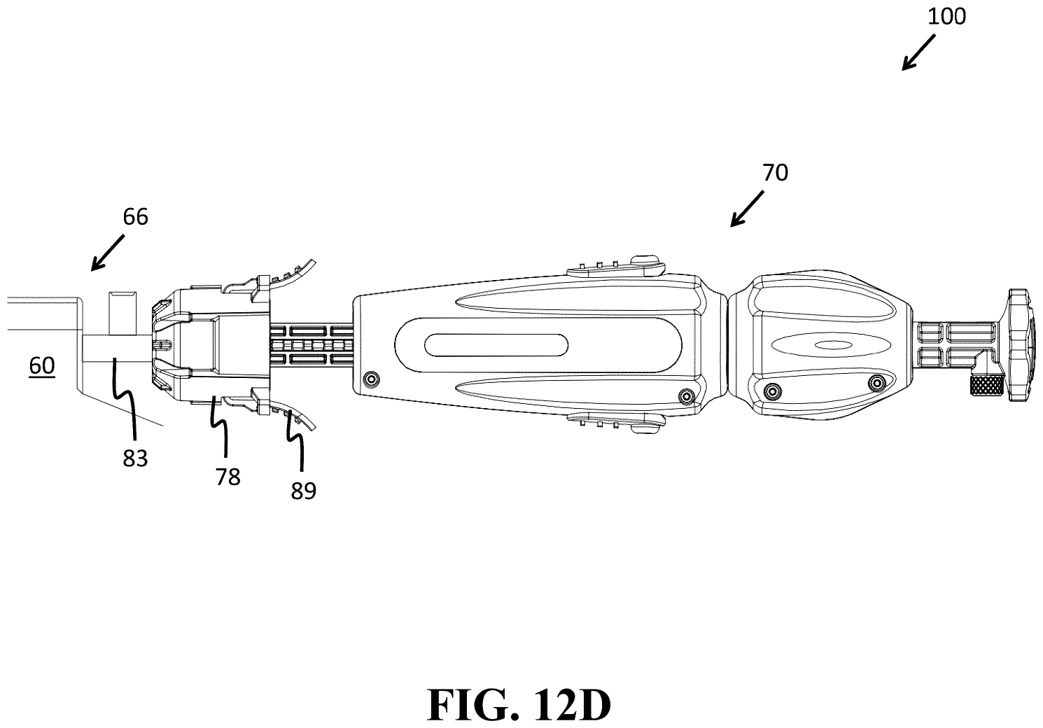

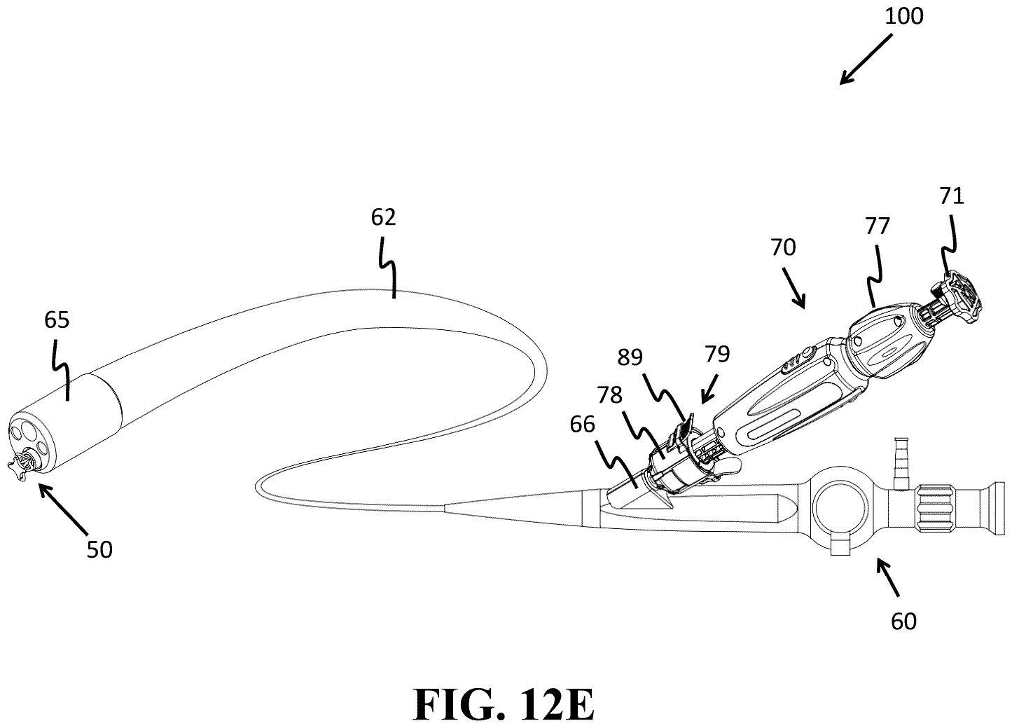

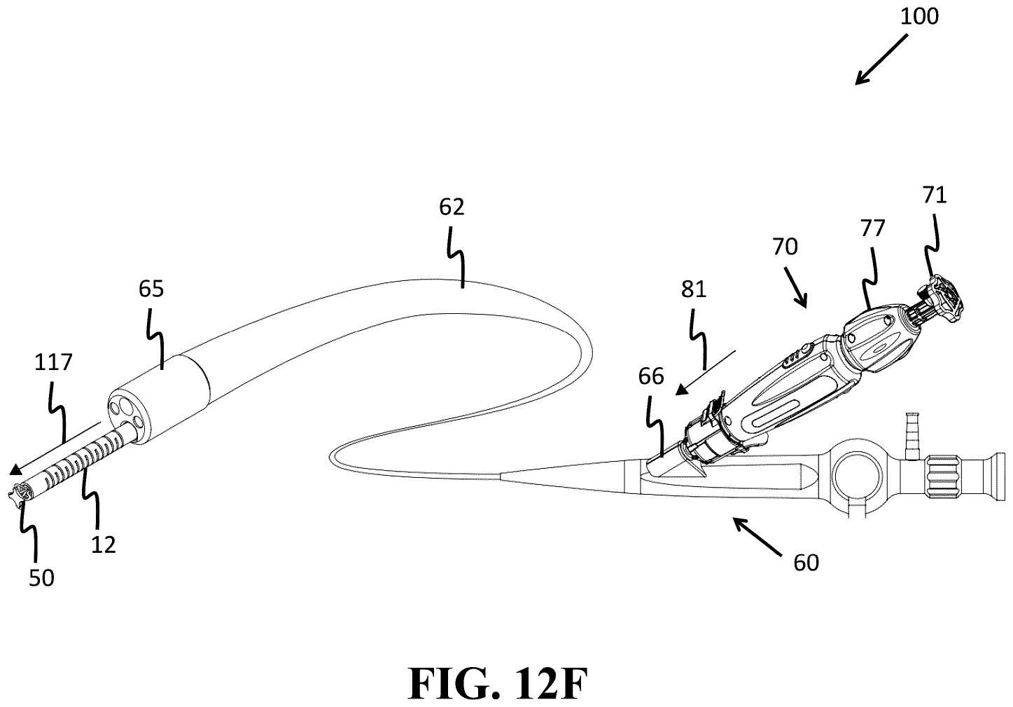

Referring now to FIGS. 12A-12F, the user interface 70 of the apparatus 100 is shown to include a quick release mechanism 79, which may be selectively coupled with the ureteroscope working channel mount 78, according to some embodiments of the present disclosure.

In some embodiments, and as depicted with particular reference to FIGS. 12B and 12C, the ureteroscope working channel mount 78 is attached to the working port 66 of the ureteroscope 60 (by moving the ureteroscope working channel mount 78 in a direction 81) prior to attachment of the user interface 70 to the ureteroscope 60. For example, the ureteroscope working channel mount 78 is first attached to an input 83 of the working port 66.

In some embodiments, and as depicted with particular reference to FIGS. 12D and 12E, in order to attach the user interface 70 to the ureteroscope 60, the tube assembly 10 may be fed through ureteroscope working channel mount 78 into the working port 66. In some embodiments, the ureteroscope working channel mount 78 includes a rubber o-ring or sealing surface which creates a water-tight seal against the second flexible tube 26 of the steerable sheath 12 via an interference fit once the steerable sheath 12 of the tube assembly 10 is inserted, to prevent backflow of irrigation.

In some embodiments, and as depicted with particular reference to FIGS. 12F, the components of the user interface 70 proximal to the ureteroscope working channel mount 78 (including the quick release mechanism 79) may be pressed into the ureteroscope working channel mount 78 (in the direction 81). As shown, this may result in the steerable sheath 12 projecting out of the distal end 65 of the outer tube 62 of the ureteroscope 60. Additionally, this may result in the quick release mechanism 79 selectively engaging the ureteroscope working channel mount 78.

In some embodiments, the quick release mechanism 79 includes flexure features and retaining surfaces which allow the proximal components of the user interface 70 to be pressed into and seated within the ureteroscope working channel mount 78, and the retaining surfaces prevent such components of the user interface 70 from being pulled out. For instance, in order to remove the proximal components of the user interface 70 from the ureteroscope working channel mount 78, release tabs 89 of the quick release mechanism 79 may be depressed which removes the retaining surface constraint, thereby allowing the proximal components of the user interface 70 to be pulled out.

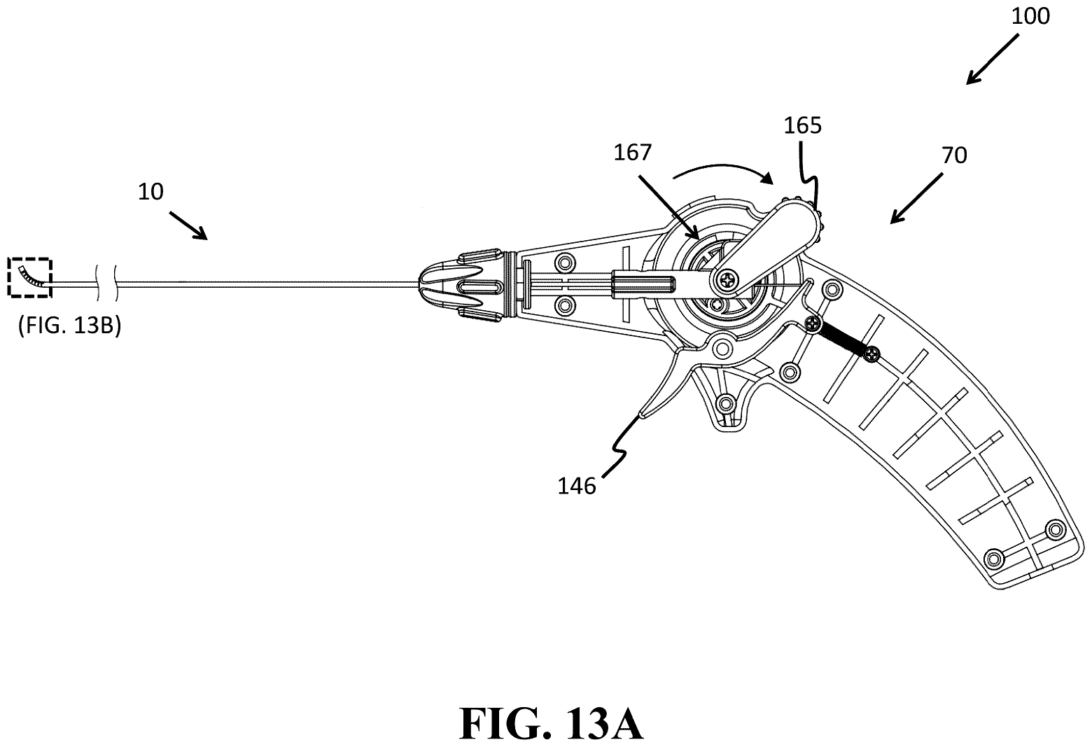

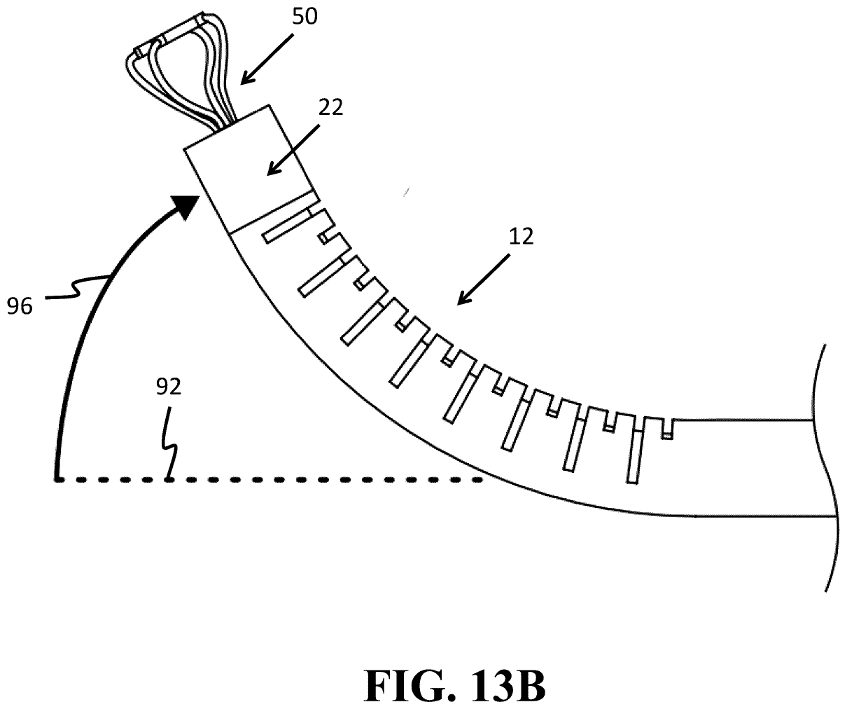

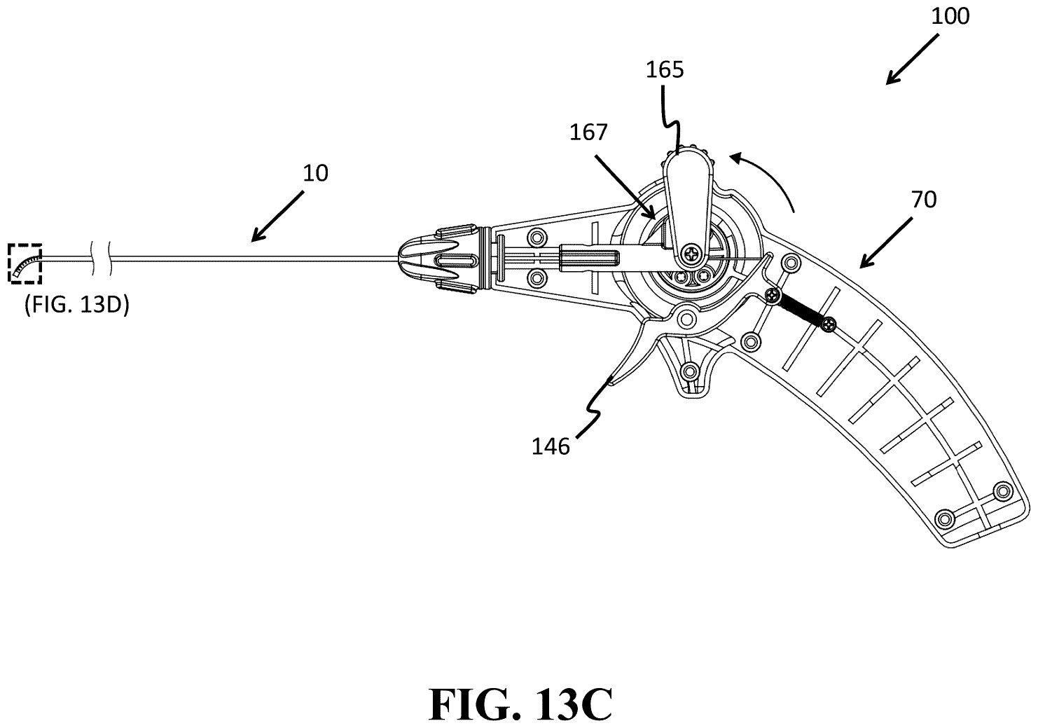

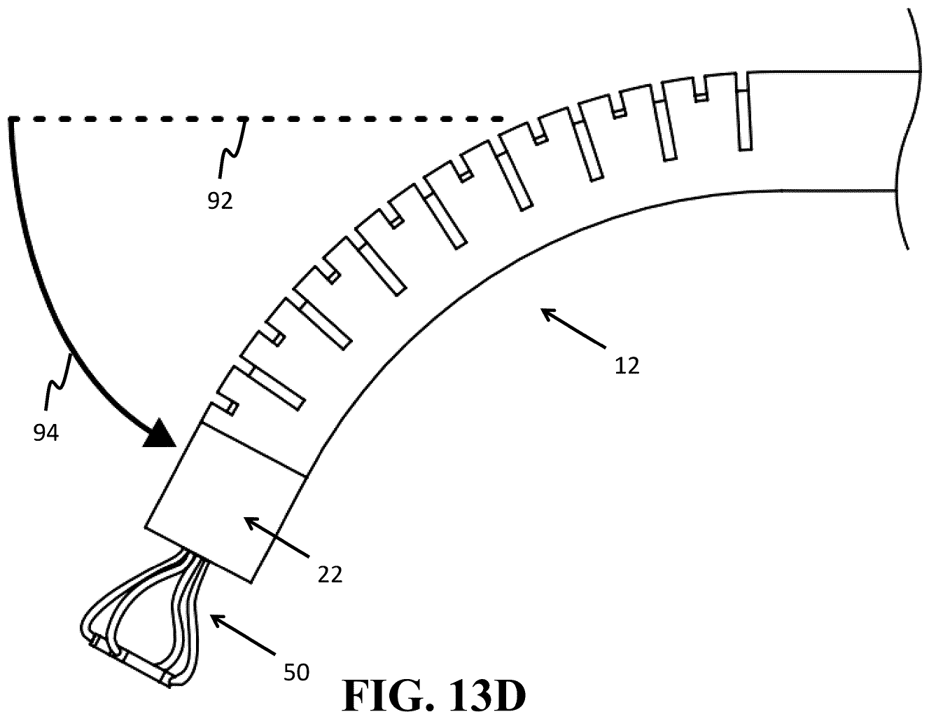

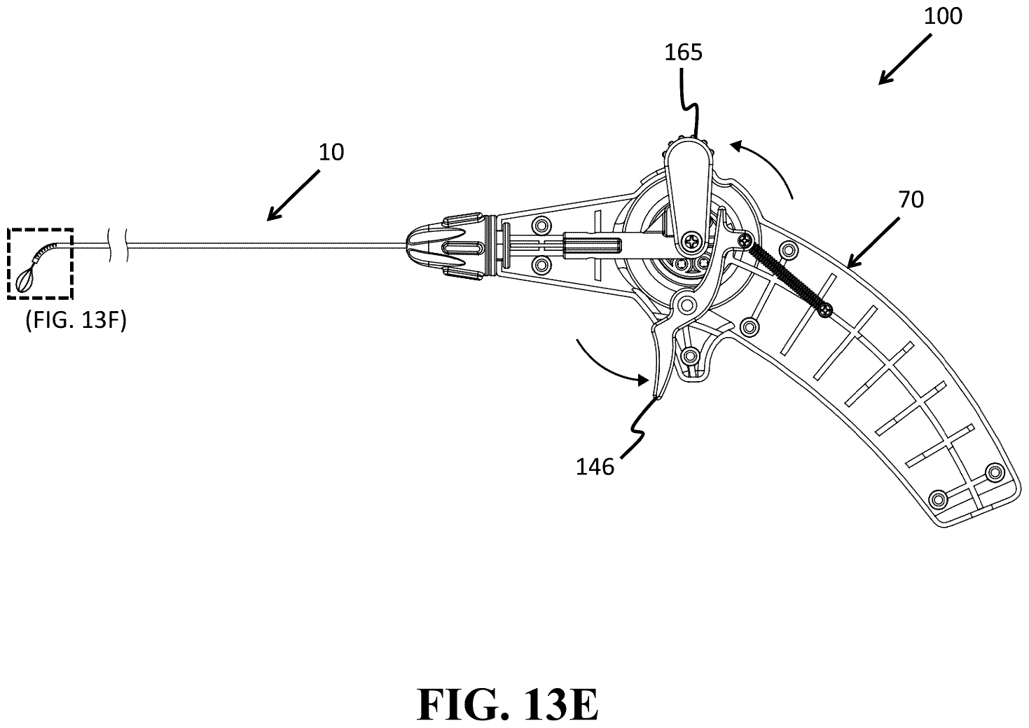

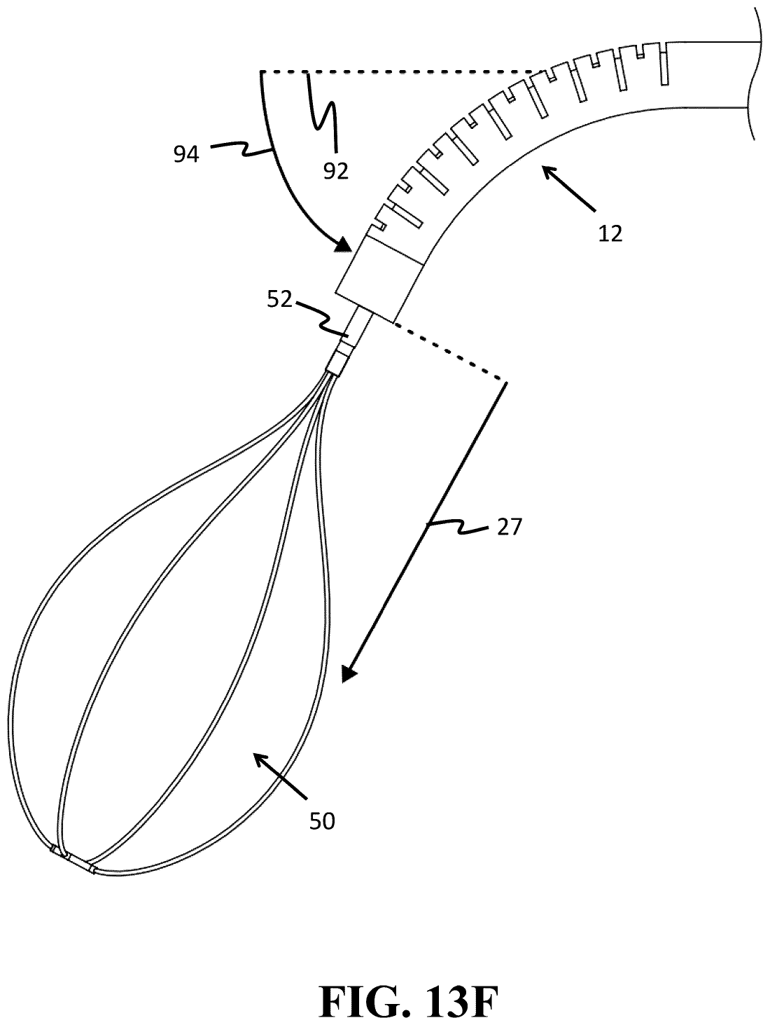

Referring now to FIGS. 13A-13F, the user interface 70 of the apparatus 100 is shown, according to some alternative embodiments of the present disclosure. In some embodiments, the user interface 70 includes in a lever 165, which may be thumb-actuated. The lever 165 may be used to impart the axial translation of the first and second flexible tubes 28, 26 relative to one another (as discussed in greater detail above), resulting actuation of the steerable sheath 12 to form a bend, thereby steering the basket 50 towards an anatomical region. As shown, the lever 165 drives a rotary-to-linear mechanism (e.g., a scotch-yoke mechanism, slider-crank mechanism, etc.) 167 to generate the required axial translation of the first and second flexible tubes 28, 26 relative to one another. As a first example, when the lever 165 is rotated clockwise as shown with reference to FIG. 13A, the steerable sheath 12 is actuated to form a bend, thereby steering the distal end 22 of the steerable sheath 12 along the path 94 as shown with particular reference to FIG. 13B. As a second example, when the lever 165 is rotated counter-clockwise as shown with particular reference to FIG. 13C, the steerable sheath 12 is actuated to form a bend, thereby steering the distal end 22 of the steerable sheath along the path 96 as shown with particular reference to FIG. 13D. Accordingly, in some embodiments, the apparatus includes a mechanical transmission that converts rotary motion to linear, or axial, translation between the first flexible tube 28 and the second flexible tube 23, such that the steerable tool 20 may be actuated to form a bend.

In some embodiments, the user interface 70 further includes a trigger 146, which may be trigger-finger actuated. The trigger 146 may be used to expand or retract the basket 50 into the released or collapsed states as discussed above. As shown with reference to FIGS. 13E and 13F, the trigger 146 may be biased by a tension spring and advance the control wire 52 into the steerable sheath 12 when depressed, causing the basket 50 to project out from the distal end 22 of the steerable sheath 12 and expand into the released state. When the trigger 146 is released, the tension spring may retract the control wire, thereby retreating the basket 50 into the distal end 22 of the steerable sheath 12, thereby forming the collapsed state of the basket 50.

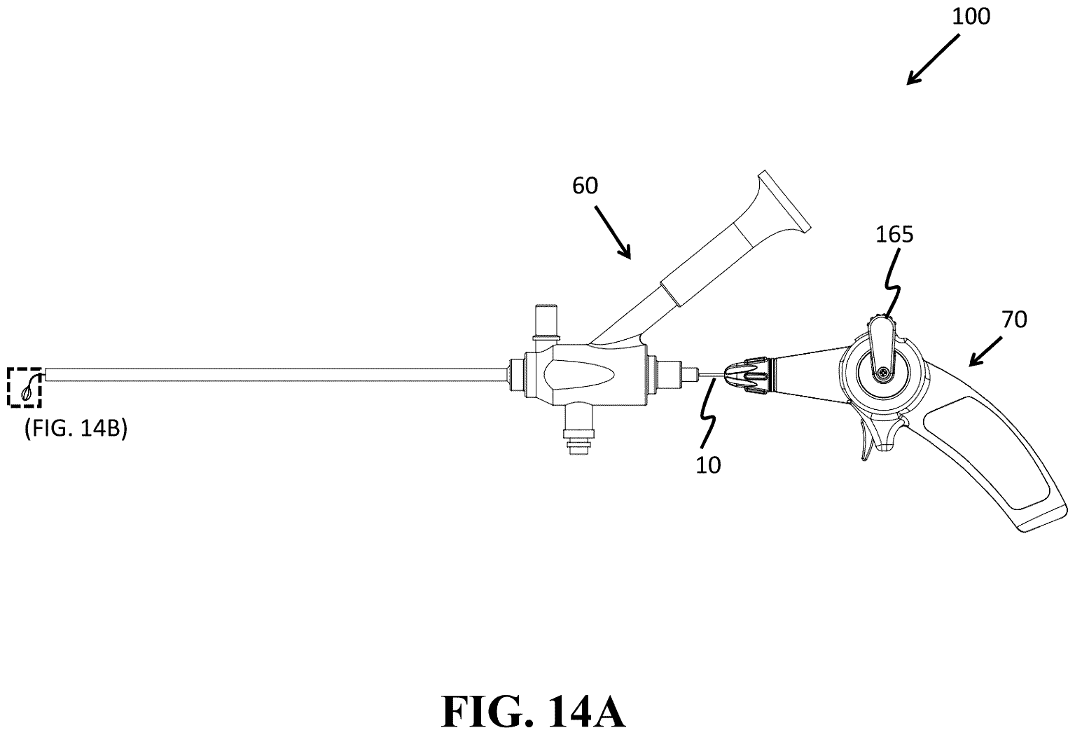

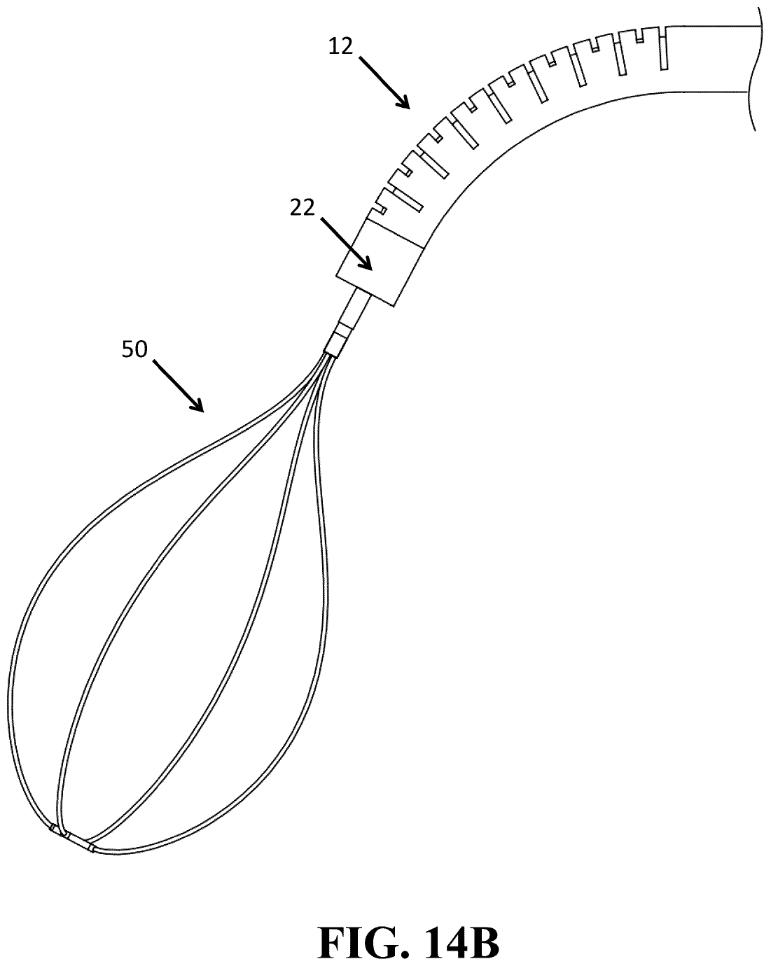

Referring now to FIGS. 14A and 14B, the ureteroscope 60 of the apparatus 100 may be a rigid ureteroscope, according to some alternative embodiments of the present disclosure. For instance, there are certain cases within urology where the tube assembly 10 may be useful through rigid or semi-rigid endoscopic platforms. Percutaneous Nephrolithotomy (PCNL) is a surgical kidney stone removal procedure where a rigid endoscope is introduced into the kidney via an incision in the patient's back, and a suite of tools (lasers, lithotripters, and baskets) are introduced through the rigid endoscope to remove a stone, such as the stone 88.

In such embodiments, the transmission section 16 of the steerable sheath 12 may feature high axial, torsional, and bending stiffness. In other words, the transmission section 16 of the steerable sheath 12 may be rigid, solid tubing, as discussed above with reference to FIG. 5A. In such embodiments, the length of the transmission section 16 may be long enough to traverse the rigid endoscope through which the tube assembly 10 is to be passed.

In other embodiments, passive flexibility along the transmission section 16 may be desirable to allow for accidental misalignment between the steerable basket and the rigid endoscope through which it is passed. This can be achieved through the inclusion of the flexible section 19 (as shown with reference to FIGS. 5B and 5C) along part or all of the transmission. In such embodiments, passive flexibility may be achieved by laser-machining a series of alternating slots, where each row of slots is rotated from the previous row by ninety degrees. The flexibility of the flexible section 19 can be modified through careful selection of the spacing between the slots (i.e. the pitch), as well as the arc length of each slot around the tube's circumference (i.e. the cut fraction). In some embodiments, the angle between subsequent slot rows may differ from ninety degrees, causing successive slots to “twist” around the longitudinal axis 92 of the steerable sheath 12.

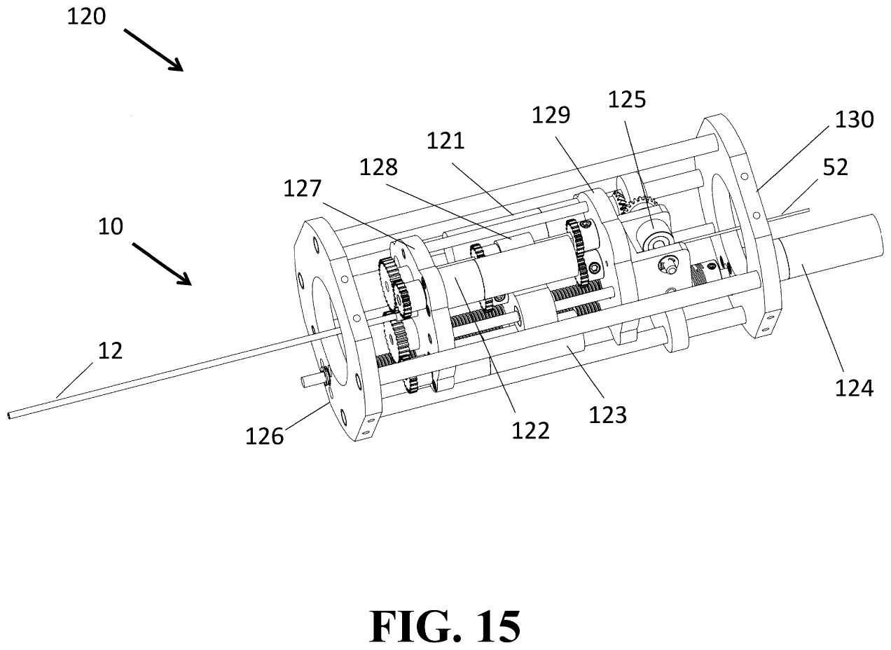

Referring now to FIG. 15, the user interface 70 of the apparatus 100 may include a robotic actuator mechanism 120, according to some embodiments of the present disclosure. For instance, the various actuations provided by the user interface 70 discussed above may be accomplished via the robotic actuator mechanism 120. The robotic actuator mechanism 120 may be controlled by a controller that is communicably coupled with the robotic actuator mechanism 120 via a wired or wireless connection. The robotic actuator mechanism 120 may be a component of the user interface 70, or be used in the apparatus 100 as an alternative to the user interface 70, depending on the implementation. For example, in the exemplary embodiment shown, the robotic actuator mechanism 120 is depicted as interacting with the steerable sheath 12 and the control wire 52 of the tube assembly 10, and includes a chassis defined by a chassis front plate 126 and the chassis back plate 130. In some embodiments, the chassis front plate 126 and/or the chassis back plate may be used to couple the robotic actuator mechanism 120 to the user interface 70, such that the robotic actuator mechanism 120 may function as the actuation components of the user interface 70 discussed above. In other embodiments, the chassis front plate 126 and/or the chassis back plate 130 may be used to couple the robotic actuator mechanism 120 to the ureteroscope 60. For instance, the chassis front plate 126 and the chassis back plate 130 may be coupled to the ureteroscope handle 61, the ureteroscope hub 63, and/or the working port 66.

As an example of the operation of the robotic actuator mechanism 120, actuation of the steerable sheath 12 to form a bend may be effectuated by a leadscrew transmission of the robotic actuator mechanism 120. In this example, the first flexible tube 28 is attached to a rear fixed mount 129, and the second flexible tube 26 is attached to an actuated mount 128 that is situated between a front fixed mount 127 and the rear fixed mount 129, which are fixed with respect to each other and separated by precision steel guiderails. A steerable sheath deflection motor 123 attached to the front fixed mount 127 is coupled to a leadscrew via a spur gear transmission, and a lead-nut in the actuated mount 128 allows the actuated mount 128 to translate with respect to the front fixed mount 127 and rear fixed mount 129 as the leadscrew is rotated. Linear bearings in the actuated mount 128 slide on the aforementioned guiderails, causing a relative translation between the second flexible tube 26 and the first flexible tube 28 as the steerable sheath deflection motor 123 rotates.

As another example, rotation of the tube assembly 10 (e.g., the rotation 112) may be effectuated by the robotic actuator mechanism 120 rotating both the first flexible tube 28 and the second flexible tube 26 at the same time. This prevents a rotational offset between the first flexible tube 28 and the second flexible tube 26 which may interfere with the deflection of the steerable sheath tube 12. In this example, a steerable sheath rotation motor 122 attached to the front fixed mount 127 is coupled to a square shaft via a spur gear transmission. This square shaft transmits rotation to driving spur gears attached to both the actuated mount 128 and the rear fixed mount 129 (with each spur gear coupled to their respective mounts via a rotary bearing interface). Each of these driving spur gears features a square-shaped inner bore, with enough clearance to allow them to slide linearly with respect to the square shaft as the actuated mount 128 is translated. These driving spur gears interface with driven spurs on their respective mounts, with the second flexible tube 26 attached to the inner bore of the driven spur gear on the actuated mount 128, and the first flexible tube 28 attached to the inner bore of the driven spur gear on the rear fixed mount 129. As the steerable sheath rotation motor 122 actuates, thereby rotating the square shaft, both the first flexible tube 28 and the second flexible tube 26 rotate by the same degree.

As yet another example, the advancement and/or retraction of the basket 50 may be effectuated by a friction drive transmission 125. In this example, the control wire 52 is sandwiched between a motor-driven drive roller and an idler roller of the friction drive transmission 125. A basket feed motor 121 attached to the rear fixed mount 129 couples to the roller of the friction drive transmission 125 via a bevel gear transmission. In other arrangements, the robotic actuator mechanism 120 drives the projection and/or retraction of the basket 50 with a second leadscrew transmission that operates in a manner similar to the manner in which the robotic actuator mechanism 120 effectuates the deflection of the steerable sheath 12.

As yet another example still, the insertion and retraction of the tube assembly 10 in its entirety may be enabled by a insertion/retraction motor 124 that translates all of the aforementioned motors 121, 122, 123, and 124; the friction drive transmission 125; and the mounts 127, 128 and 129 with respect to a fixed outer chassis defined by a chassis front plate 126 and a chassis back plate 130. A insertion/retraction motor 124 attached to the chassis back plate 130 back drives a leadscrew via a mechanical coupling, which interfaces with a lead-nut on the rear fixed mount 129, such that a rotation applied to the leadscrew causes the rear fixed mount 129, front fixed mount 127, and actuated mount 128 to translate as a unit. Rotation is prevented via linear bearings in the front fixed mount 127 and the rear fixed mount 129 that interface with precision guiderails attached to the chassis front plate 126 and the chassis back plate 130, respectively.

Thus, although there have been described particular embodiments of the present invention of a new and useful METHOD AND APPARATUS FOR ENDOSCOPIC STONE RETRIEVAL, it is not intended that such references be construed as limitations upon the scope of this invention.

What is claimed is:

1. An endoscopic apparatus, comprising:
a steerable sheath including a first flexible tube concentrically nested within a second flexible tube;
a control wire disposed within the steerable sheath and movable therein along a longitudinal axis of the steerable sheath; and
a basket disposed on the control wire and positioned at least partially within the steerable sheath, the basket including a plurality of wires,
wherein the first flexible tube includes a first deflectable portion, the second flexible tube includes a second deflectable portion, the first and second deflectable portions being selectively weakened portions of the first and second flexible tubes that are angularly oriented, relative to a longitudinal axis of the steerable tool, in directions that are offset from each other by an angle equal to or less than one-hundred and eighty degrees,
wherein the first and second flexible tubes are joined at a location distal to the first and second deflectable portions,
wherein the steerable tool is actuable to form a bend by relative axial translation between the first flexible tube and the second flexible tube,
wherein advancing movements of the control wire cause the basket to project out of a distal end of the steerable sheath and expand, and
wherein retreating movements of the control wire cause the basket to retract into the distal end of the steerable sheath and contract.

2. The apparatus of claim 1, wherein the first and second deflectable portions each include a serpentine profile of material removed from the first and second flexible tubes.

3. The apparatus of claim 1, wherein the first and second deflectable portions each include a series of cutout sections spaced along the longitudinal axis of the steerable tool.

4. The apparatus of claim 1, wherein the plurality of wires each have a pre-formed curvature,
wherein each of the plurality of wires are coupled to each other at their distal ends, and
wherein each of the plurality of wires are coupled, at their proximal ends, to the distal end of the control wire.

5. The apparatus of claim 1, wherein the plurality of wires are coupled at their distal ends via a cylindrical member.

6. The apparatus of claim 1, wherein the plurality of wires are coupled at their distal ends via a joining disk.

7. The apparatus of claim 1, wherein the steerable sheath includes a steerable section that is actuable to form the bend, and a transmission section located further from the basket than the steerable section, and
wherein the transmission section includes a passive section and a rigid section, wherein a flexibility of the passive section is greater than a flexibility of the rigid section.

8. The apparatus of claim 7, wherein the passive section of the transmission section is located closer to the steerable section than the rigid section of the transmission section.

9. A method of performing endoscopic surgery, comprising:
providing a steerable sheath, a control wire disposed within the steerable sheath, and a basket disposed on the control wire;
forming a bend in the steerable sheath, wherein forming the bend causes a distal end of the steerable sheath to be steered toward an anatomical region within a patient;
advancing the control wire relative to the steerable sheath, wherein advancing the control wire relative to the steerable sheath causes the basket to project out of the distal end of the steerable sheath and expand, such that the basket is positioned about an object located within the anatomical region; and
retreating the control wire relative to the steerable sheath, wherein retreating the control wire relative to the steerable sheath causes the basket to retract into the distal end of the steerable sheath and contract, such that the basket retains the object.

10. The method of claim 9, further comprising advancing the steerable sheath through a urinary system of a patient.

11. The method of claim 10, wherein advancing the steerable sheath through the urinary system of the patient includes advancing the steerable sheath through a ureter of a patient and toward a calyces of the patient's kidney, such that the distal end of the steerable sheath is located in or near the calyces,
wherein the object is a kidney stone.

12. The method of claim 11, wherein the steerable sheath include a first flexible tube concentrically nested within a second flexible tube,
wherein the first flexible tube includes a first deflectable portion and the second flexible tube includes a second deflectable portion,
wherein the first and second deflectable portions are selectively weakened portions of the first and second flexible tubes that are angularly oriented, relative to a longitudinal axis of the steerable tool, in directions that are offset from each other by an angle equal to or less than one-hundred and eighty degrees, and
wherein the first and second flexible tubes are joined at a location distal to the first and second deflectable portions.

13. The method of claim 12, wherein the first and second deflectable portions each include a serpentine profile of material removed from the first and second flexible tubes.

14. The method of claim 12, wherein the first and second deflectable portions each include a series of cutout sections spaced along the longitudinal axis of the steerable tool.

15. The method of claim 12, wherein the basket includes a plurality of wires,
wherein the plurality of wires each have a pre-formed curvature,
wherein the plurality of wires are coupled to each other at their distal ends, and
wherein the plurality of wires are coupled, at their proximal ends, to a distal end of the control wire.

16. The method of claim 15, wherein the plurality of wires are coupled at their distal ends via a cylindrical member.

17. The method of claim 15, wherein the plurality of wires are coupled at their distal ends via a joining disk.

18. An endoscopic apparatus, comprising:
a steerable sheath that is actuable to form a bend;
a control wire disposed within the steerable sheath and movable therein along a longitudinal axis of the steerable sheath; and
a basket disposed on the control wire and positioned at least partially within the steerable sheath, the basket including a plurality of wires that each have a pre-formed curvature,
wherein the plurality of wires are coupled to each other at their distal ends and are coupled at their proximal ends to a distal end of the control wire,
wherein advancing movements of the control wire cause the basket to project out of the distal end of the steerable sheath and expand, and
wherein retreating movements of the control wire cause the basket to retract into the distal end of the steerable sheath and contract.

19. The apparatus of claim 18, wherein the steerable sheath includes a first flexible tube concentrically nested within a second flexible tube,
wherein the first flexible tube includes a first deflectable portion and the second flexible tube includes a second deflectable portion,
wherein the first and second deflectable portions are selectively weakened portions of the first and second flexible tubes that are angularly oriented, relative to a longitudinal axis of the steerable tool, in directions that are offset from each other by an angle equal to or less than one-hundred and eighty degrees,
wherein the first and second flexible tubes are joined at a location distal to the first and second deflectable portions, and
wherein the steerable tool is actuable to form a bend by relative axial translation between the first flexible tube and the second flexible tube.

20. The apparatus of claim 19, wherein the steerable sheath includes a steerable section that is actuable to form the bend, and a transmission section located further from the basket than the steerable section,
wherein the transmission section includes a passive section and a rigid section, wherein a flexibility of the passive section is greater than a flexibility of the rigid section, and
wherein the passive section of the transmission section is located closer to the steerable section than the rigid section of the transmission section.
